(19) United States
(12) Patent Application Publication
(10) Pub. No.: US 2006/0100578 A1
(43) Pub. Date: May 11, 2006

(54) Medication delivery apparatus and methods for intravenous infusions

(75) Inventor: Marc S. Lieberman, Poway, CA (US)

Correspondence Address: FOLEY & LARDNER LLP, P.O. BOX 80278, SAN DIEGO, CA, 92138-0278, US

(73) Assignee: TANDEM MEDICAL, INC.

(21) Appl. No.: 10/939,979

(22) Filed: Sep. 13, 2004

(51) Int. Cl. A61M 37/00; A61M 31/00

(52) U.S. Cl. 604/132; 604/507

(57) ABSTRACT

The present invention relates to delivery containers designed to deliver fluids for infusion to patients in a predetermined sequence, and methods for their construction and use. The containers described herein integrally comprise a plurality of non-fluidly connected chambers. The containers may be configured to deliver a volume of each medication of an infusion therapy in a predetermined sequence, duration, and/or interval from these chambers; alternatively, a container may be part of a larger device that provides the necessary hardware to perform such predetermined delivery. The container provides improved infusion therapy administration by reducing opportunities for error, infection, adverse drug interactions, or other complications.

### RELATED APPLICATIONS

This application is a continuation-in-part of U.S. patent application Ser. No. 10/111,587, filed Apr. 24, 2002 (pending), which is a 371 of PCT/US00/41860, filed Nov. 2, 2000, which claims priority to U.S. patent application Ser. No. 09/434,975, filed Nov. 5, 1999 (and has issued as U.S. Pat. No. 6,428,518). The present application also claims priority to U.S. patent application Ser. No. 10/251,491, filed Sep. 19, 2002, which, in turn, claims priority to U.S. Provisional Patent Application No. 60/337,407, filed Dec. 3, 2001 (abandoned); to U.S. patent application Ser. No. 09/713,521, filed Nov. 14, 2000 (pending), which is a divisional of U.S. patent application Ser. No. 09/231,535, filed Jan. 14, 1999, which issued as U.S. Pat. No. 6,146,360, which is a continuation of U.S. patent Ser. No. 09/008,111, filed Jan. 16, 1998, which issued as U.S. Pat. No. 6,074,366; to U.S. patent application Ser. No. 09/434,974, filed Nov. 5, 1999, which issued as U.S. Pat. No. 6,669,668; and to U.S. patent application Ser. No. 09/434,972, filed Nov. 5, 1999, which issued as U.S. Pat. No. 6,726,555; each of which is hereby incorporated by reference in their entirety, including all tables, figures, and claims.

### FIELD OF THE INVENTION

The present invention relates to apparatus and methodology for the delivery, e.g., intravenous infusion, of medication and/or other fluids in accordance with a predetermined medical therapy. More particularly, the present invention relates to medication delivery apparatus and methodology with improved ease of administration of a variety of therapeutic agents by intravenous infusion.

### BACKGROUND OF THE INVENTION

Intravenous medications including antibiotics and the like may be administered intermittently over an extended period of time. Each administration of an intravenous therapy generally follows a predefined procedure that often includes a series of manual steps. Such manual steps may include saline flushes and generally terminate with the application of anti-clotting medication. The manual steps in the therapy procedures are a principle source of error, infection, and other complications that may arise during intermittent infusion therapy.

Examples of medication delivery containers and medication delivery pumps have been described in U.S. Pat. No. 6,146,360; U.S. Pat. No. 6,074,366; U.S. patent application Ser. No. 09/434,972, filed on Nov. 5, 1999; and U.S. patent application Ser. No. 09/434,974, filed on Nov. 5, 1999; each of which is hereby incorporated by reference in its entirety, including all tables, figures, and claims.

Accordingly, there is still a need in the art for apparatus and methodology which improve the administration of intermittent medication infusion therapy. The present invention satisfies these and other needs in the art.

### BRIEF DESCRIPTION OF THE INVENTION

The present invention overcomes many of the problems in the art by providing medication delivery containers designed to deliver fluids in a predetermined sequence, and methods for their construction and use. The containers described herein comprise a plurality of non-fluidly connected chambers that are integral to the container. The phrase “integral container” is defined hereinafter. The integral containers of the present invention may be configured to deliver a volume of each fluid in a selected infusion regimen in a predetermined sequence, duration, volume, and/or interval from these chambers. Alternatively, a container may be part of a larger device that provides additional hardware to perform the desired sequential delivery. The container provides improved infusion therapy administration by reducing opportunities for error, infection, adverse drug interactions, or other complications.

In various embodiments, fluids may be delivered from the integral containers of the present invention by application of positive pressure to one or more non-fluidly connected chambers, by application of negative pressure to one or more non-fluidly connected chambers, by gravity feed from one or more non-fluidly connected chambers, or by some combination of these delivery modes.

In certain preferred embodiments, positive pressure is created by compression of a chamber within the integral containers of the present invention, thus expressing fluid from that chamber through a port in the chamber wall. In these embodiments, the chamber is preferably flexible, and positive pressure may be generated in a plurality of chambers in a predetermined sequence, for example, by a roller pump compressing each chamber at the proper time, thereby delivering the fluids from the integral container in the desired sequence. Other means of generating positive pressure, such as injection of a gas or other fluid into a chamber to express some or all of the contents of that chamber, are also contemplated by the present invention.

In other preferred embodiments, negative pressure is created by application of a pump to an output port or conduit fluidly connected to a chamber within the integral containers of the present invention, thereby extracting fluid from that chamber through a port in the chamber wall.

Controlled fluid flow from the integral containers of the present invention may be obtained using a variety of methodologies. For example, force (either positive, negative, or gravity) may be used to deliver fluid from one or more chambers within the integral container in sequence. This may be achieved, e.g., by allowing a single pump to access a plurality of chambers in sequence; or by having multiple pumps, each of which may be connected to a corresponding chamber, and actuating the pumps in sequence. Alternatively, valves that control flow from each chamber may be actuated (manually, electronically, pneumatically, etc.) in sequence, thereby permitting flow to occur from a given chamber. The skilled artisan will understand that such control means need not be selected individually, and that a given device might include control at both the pump and valve level for example.

In certain preferred embodiments, fluid flows from a plurality of chambers to a manifold that receives flow from several input conduits, and that generates flow through a single exit conduit. The functionality of a manifold may also be served by other flow structures, such as a set of multi-path (e.g., three-way) valves or connections placed in series. In such embodiments, each multi-path connection might receive flow from a previous chamber or valve, as well as from a new chamber, with a resulting flow to a single exit conduit or port.

In certain preferred embodiments, the integral containers of the present invention may be constructed as a flexible bag having a plurality of non-fluidly connected chambers. Such containers may also include structures for minimizing pressure drop which may be associated with a chamber upon the application of pressure to the respective chamber, thereby allowing relatively unimpeded fluid flow from the respective chamber to an associated conduit during the application of pressure to the chamber.

While the present invention relates in part to containers that may be provided to a medical provider (e.g., physician or pharmacist) or other user in an unfilled state for subsequent filling in a manner deemed appropriate by that user, in various aspects the invention also relates to containers in which one or more, and preferably all, chambers within the container are provided to a user pre-filled with fluids to be delivered in a predetermined sequence.

In accordance with additional embodiments of the present invention, there are provided medication delivery systems comprising a bag having at least one chamber containing a medication fluid and a manifold, and a pump having an activating mechanism configured to activate the chamber(s) to dispense the fluid from the bag.

In accordance with further embodiments of the present invention, there are provided medication delivery containers comprising a bag having a plurality of chambers, and a manifold assembly coupled to the plurality of chambers for delivering medications out of the chambers.

In accordance with still further embodiments of the present invention, there are provided fluid delivery containers comprising a bag having at least one fluid chamber and structure for minimizing pressure drop between the chamber and an associated conduit upon the application of pressure to the chamber.

In accordance with additional embodiments of the present invention, there are provided fluid delivery containers for the automated infusion of a plurality of pharmacological agents, wherein the container comprises a plurality of chambers each configured with a respective geometry for controlling the administration of the plurality of pharmacological agents. The container additionally comprises a manifold assembly having a plurality of valves for controlling the administration of the plurality of pharmacological agents to an infusion site. Each chamber of the fluid delivery container has a configuration that controls the volume of each pharmacological agent administered and the regimen with which said pharmacological agent is administered.

In accordance with further embodiments of the present invention, there are provided fluid delivery pumps comprising a structure for sequentially applying constant force to compress a flexible fluid container from a first end towards a second end of said container; and an energy absorption device coupled to the structure for sequentially applying constant force for limiting the maximum rate at which said structure compresses the fluid container.

In accordance with still further embodiments of the present invention, there are provided charging disks comprising first and second spring-loaded pawls, the first pawl having a shaft that engages a slot in the second pawl, the shaft and slot being configured such that the second pawl is depressed when the first pawl is depressed, but the first pawl is not depressed when the second pawl is depressed.

In accordance with additional embodiments of the present invention, there are provided methods for filling an invention fluid delivery bag having a plurality of chambers. In the invention methods, a first predetermined fluid volume is measured; at least one chamber of the bag is constrained to a second predetermined volume; and the plurality of chambers are filled through a bulk fill port with the first predetermined volume of fluid such that a constrained chamber is filled with the second predetermined volume of fluid. A remaining chamber is then filled with the first predetermined volume of fluid minus the fluid of the constrained chamber.

In accordance with further embodiments of the present invention, there are provided methods for delivering medication fluids. Invention fluid delivery methods comprise compressing a bag having at least one chamber containing a medication fluid using a constant force spring to generate a predetermined pressure in the chamber based on the chamber's configuration and delivering the medication fluid from the bag at the predetermined pressure to an infusion site using a micro-bore tubing having a length and an inner diameter that establishes a predetermined flow rate.

In accordance with still further embodiments of the present invention, there are provided methods for charging an infusion pump having a constant force spring coupled to first and second cover doors by a charging assembly. The invention charging method comprises opening the first cover door to partially charge the constant force spring; and opening the second cover door to fully charge the constant force spring.

The foregoing summary of the invention is non-limiting, and other features of the invention will be apparent to those of skill in the art from the following figures, detailed description of the invention, and the claims.

### BRIEF DESCRIPTION OF THE FIGURES

FIG. 1 is a schematic view of an integral container according to the invention, showing a plurality of non-fluidly connected chambers connected to a set of valves in parallel (A), in series (B), and in combination of the two (C).

FIG. 2 is a schematic view of an integral container according to the invention, showing a plurality of non-fluidly connected chambers connected to a set of valves in parallel. The figure shows two possible locations for valves (i.e., within the integral container itself, or within an external flow path leading from each chamber), together with possible locations for an accumulator chamber.

FIG. 3 is a schematic view of an integral container according to the invention, showing a plurality of non-fluidly connected chambers connected to a set of actively-controlled valves and single pump to generate flow using negative pressure.

FIGS. 4 and 5 collectively show an exemplary pumping sequence, using a shuttle valve to provide sequential delivery from each chamber in an integral fluid delivery container.

FIG. 6 is a perspective view of an exemplary medication delivery container according to the invention.

FIG. 7 is a plan view of the medication delivery container of FIG. 6.

FIG. 8 is a plan view of a multi-chamber bag of the medication delivery container of FIG. 6, showing the bag's chambers and conduits and one embodiment of a chamber flex absorbing pattern.

FIG. 9 is a cross-sectional view along line A-A of a multi-chamber bag of FIG. 8.

FIG. 10 is a plan view of a multi-chamber bag of the medication delivery container of FIG. 6, showing an alternate embodiment of the chamber flex absorbing pattern.

FIG. 11 is a plan view of a multi-chamber bag of the medication delivery container of FIG. 6, showing yet another embodiment of the chamber flex absorbing pattern.

FIG. 12 is a perspective view of a manifold assembly of the medication delivery container of FIG. 6.

FIG. 13 is a perspective view of the manifold assembly of FIG. 12 from a reverse direction.

FIG. 14 is an exploded perspective view of the manifold assembly of FIG. 12.

FIG. 15 is a schematic diagram showing the internal conduit and valve configuration of the manifold assembly of FIGS. 12-14.

FIG. 16 is a perspective view of an exemplary medication delivery pump according to the present invention.

FIG. 17 is a perspective view of the medication delivery pump of FIG. 16, with the pump's cover doors in a fully opened position.

FIG. 18 is an exploded perspective view of the medication delivery pump of FIG. 16.

FIG. 19 is a perspective view of a spring assembly of the medication delivery pump of FIG. 16.

FIG. 20 is an exploded perspective view of the spring assembly of FIG. 19.

FIG. 21 is a perspective view of a constant force spring, in a stretched position, of the spring assembly of FIG. 19.

FIG. 22 is a plan view of the constant force spring of FIG. 21, in a stretched position.

FIG. 23 is an elevation view of the constant force spring of FIGS. 21 and 22.

FIG. 24 is an exploded perspective view of a base assembly of the medication delivery pump of FIG. 16.

FIG. 25 is a perspective view of a gear box assembly of the medication delivery pump of FIG. 16.

FIG. 26 is an exploded perspective view of the gear box assembly of FIG. 25.

FIG. 27 is an exploded perspective view of the energy absorption device shown in the gear box assembly of FIG. 25.

FIG. 28 is an elevation view of an energy absorption device shown in the gear box assembly of FIG. 25.

FIG. 29 is a cross-sectional side elevation view of the medication delivery pump of FIG. 16 taken through the middle of the pump.

FIG. 30 is an elevation view of the medication delivery pump of FIG. 16 with a side cover removed, showing the position of a charging disk, the spring assembly and the pump's cover doors with the spring in a fully coiled or uncharged position.

FIG. 31 is an elevation view of the medication delivery pump of FIG. 16 with a side cover removed, showing the position of the charging disk, the spring assembly and the pump's cover doors with the spring in a half-coiled or half-charged position.

FIG. 32 is an elevation view of the medication delivery pump of FIG. 16 with a side cover removed, showing the position of the charging disk, the spring assembly and the pump's cover doors with the spring in a three-fourths uncoiled or three-fourths charged position.

FIG. 33 is an elevation view of the medication delivery pump of FIG. 16 with a side cover removed, showing the position of the charging disk, the spring assembly and the pump's cover doors with the spring in a fully uncoiled or charged position.

FIG. 34 is a partially exploded perspective view of the charging disk of the medication delivery pump of FIG. 16, having spring loaded pawls.

FIG. 35 is a partial cross-sectional view of the medication delivery pump of FIG. 16 showing forces (as arrows) of a constant force spring upon the medication-containing bag.

FIG. 36 is a plan view of a spring guard of the medication delivery pump of FIG. 16.

FIG. 37 is an elevation view of the spring guard of FIG. 36.

FIG. 38 is a schematic view of an exemplary administration set suitable for use in the medication delivery system of the invention.

FIG. 39 is a perspective view of the medication delivery bag placed in a receptacle area of the housing of the medication delivery pump.

FIG. 40 is a graph showing fluid flow rate, versus time, from chambers 1-4 of a medication delivery bag in accordance with the medication delivery system of the invention.

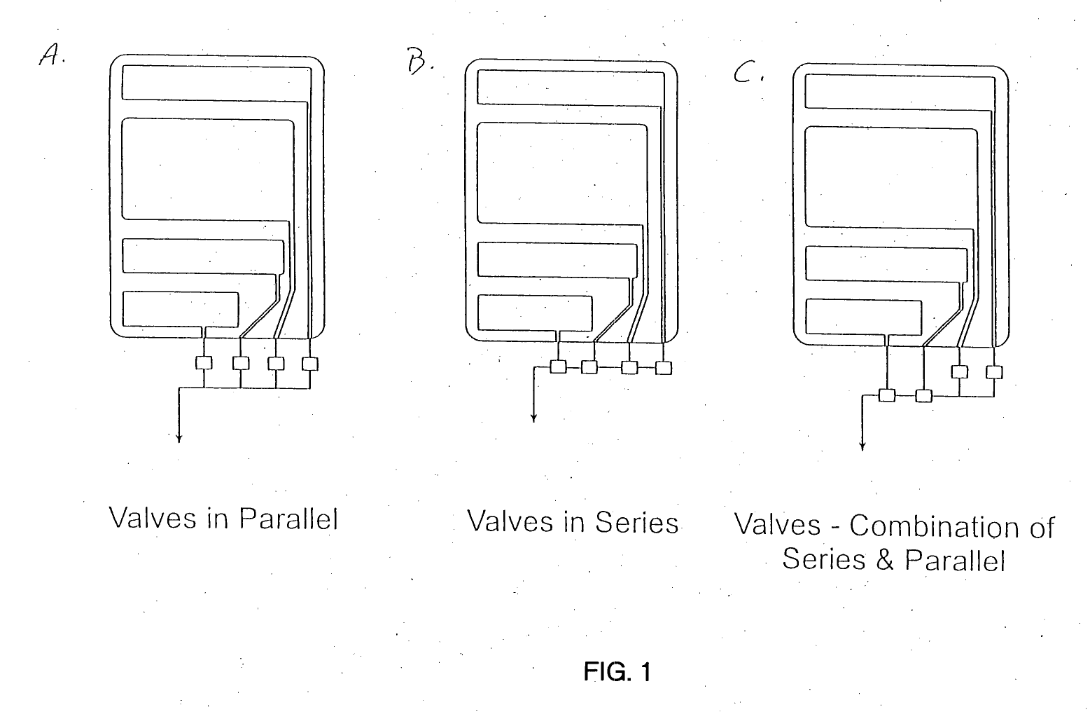

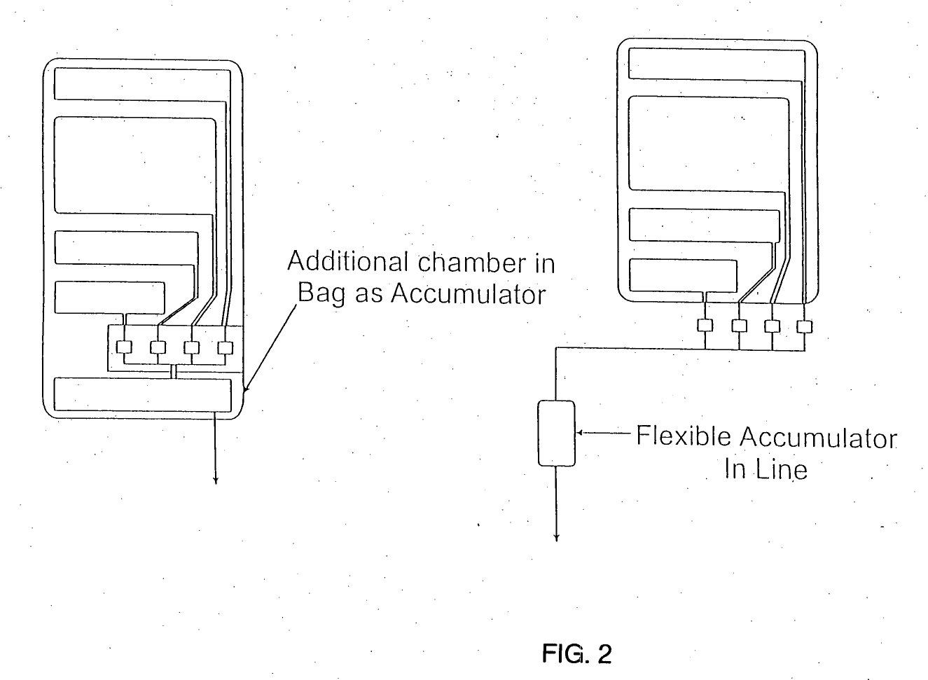

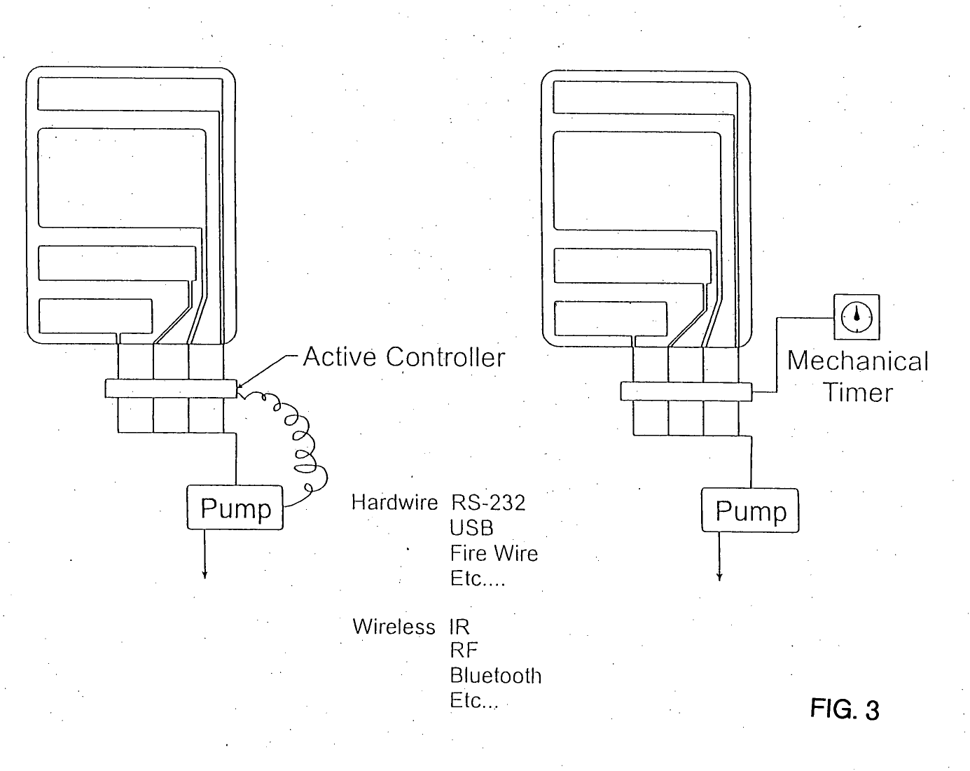

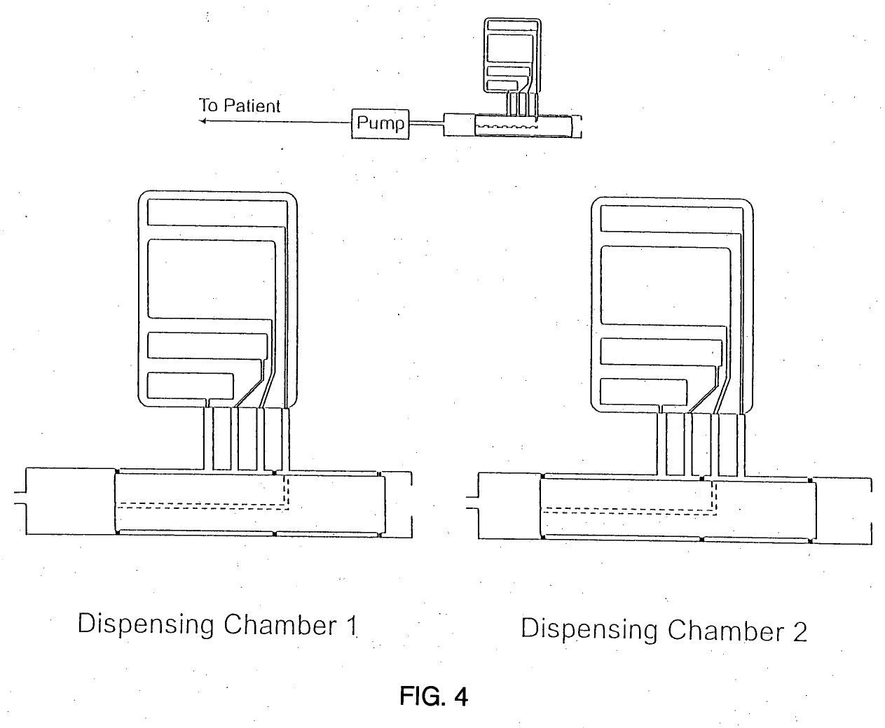

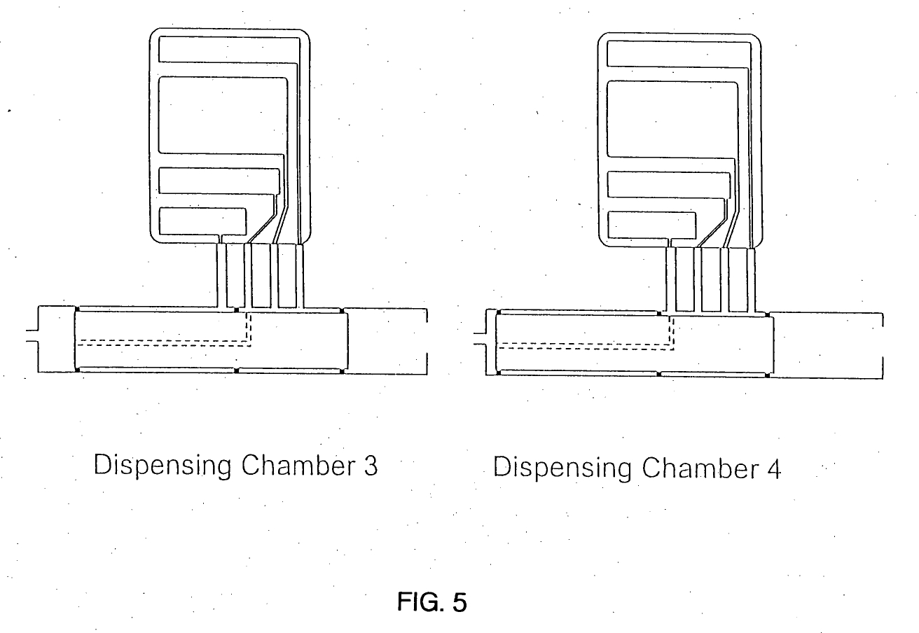

### DETAILED DESCRIPTION OF THE INVENTION

In accordance with the present invention, there are provided medication delivery containers designed to deliver fluids in a predetermined sequence, and methods for the construction and use thereof. The containers described herein comprise a plurality of non-fluidly connected chambers that are integral to the container. The containers may be configured to deliver a volume of each fluid of an infusion therapy regimen in a predetermined sequence, duration, volume and/or interval from these chambers; alternatively, a container may be part of a larger device that provides the necessary hardware to perform such sequential delivery.

Fluids may be delivered from the non-fluidly connected chambers by gravity, by the generation of positive pressure within a chamber, by the generation of negative pressure within a chamber, or by a combination of the above. Control of this fluid flow may be obtained by careful configuration of the geometry of the chambers and conduits within the container, by controlled pump actuation, by controlled valve actuation, or by a combination of such control means.

The phrase “integral container” as used herein refers to a container comprising a plurality of non-fluidly connected chambers, in which removal of a chamber would result in a loss of integrity of the entire container. For example, a preferred embodiment of the integral containers of the present invention is a flexible bag in which the chamber walls are formed from the container walls. Thus, in these embodiments, removal of a chamber would also entail removal of a portion of the container itself. By way of contrast, U.S. Pat. No. 5,658,271 discloses a device in which individual containers are placed in a housing. In this non-integral container, each bag may be replaced without disrupting the integrity of the larger housing.

The phrase “non-fluidly connected” as used herein in reference to chambers within an integral container refers to an absence of fluid connections between the chambers themselves that would allow fluids to intermingle before flowing from one of the chambers to a patient. Such chambers may intermingle fluids at a point downstream from the chambers, such as at a manifold, but the chambers from which the fluids originate would still be non-fluidly connected. Additionally, such chambers may be connected, such as via a conduit, but so long as no fluids to be delivered from each chamber to a patient intermingle before their delivery out of the chambers, the chambers would still be said to be non-fluidly connected.

The phrase “substantially non-fluidly connected” as used herein in reference to chambers within an integral container refers to chambers in which fluid connections between the chambers allow less than 10% of fluids from one chamber to intermingle with the fluids in another chamber before flowing from one of the chambers to a patient. More preferably, chambers that are substantially non-fluidly connected allow less than 5%, and most preferably less than 1%, of fluids from one chamber to intermingle with the fluids in another chamber before flowing from one of the chambers to a patient.

The phrase “fluidly connected” as used herein in reference to chambers within an integral container refers to chambers in which fluid connections between the chambers allow 10% or more of the fluids from one chamber to intermingle with the fluids in another chamber before flowing from one of the chambers to a patient. Such fluidly connected chambers may originate as non-fluidly connected or substantially non-fluidly connected chambers, but be rendered fluidly connected prior to delivery of fluids from one of the chambers. For example, a frangible seal between chambers may be breached, allowing the fluids in the chambers to intermingle. Preferably, fluidly connected chambers allow 50% or more, and most preferably 90% or more, of the fluids from one chamber to intermingle with the fluids in another chamber before flowing from one of the chambers to a patient.

The phrase “predetermined sequence” refers to delivery of a plurality of fluids from an integral container to a patient according to a treatment regimen desired by a clinician. Such a predetermined sequence may involve delivery of fluids discretely, i.e., a first fluid is completely delivered before a second fluid is delivered, or in an overlapping manner, i.e., all or a portion of a second fluid is delivered at the same time that a first fluid is delivered. The predetermined sequence may include both controlled timing of delivery, volume of delivery, and/or order of delivery.

The phrase “positive pressure” as used herein refers to the application of force to a fluid or chamber resulting in fluid pressure within a chamber that is greater than the force of gravity; that is, a pressure greater than that created by the hydrostatic head pressure within the chamber. Such positive pressures may be generated by a pump or other means of pushing on a fluid or chamber. Suitable positive pressures are any pressure that the chamber may withstand without breaching the integrity of the chamber (e.g., bursting). Preferred pressures are between 100 psi and 0.1 psi, more preferably between 40 psi and 0.5 psi, and most preferably between 10 psi and 1 psi.

Similarly, the phrase “negative pressure” refers to the application of force to a chamber resulting in fluid pressure within a chamber or conduit that is less than the force of gravity. Such pressures are often referred to as “suction pressures” or “vacuum pressures.” Negative pressures may be generated by a pump or other means of pulling fluid from chamber. Suitable negative pressures are any pressure that the chamber, or any conduit between the chamber and the source of the negative pressure, may withstand without collapsing. Preferred pressures are between 5 psi and 0.1 psi, more preferably between 3 psi and 0.25 psi, and most preferably between 1 psi and 0.5 psi.

The phrase “gravity feed” refers to the use of only gravitational forces to deliver a fluid. The skilled artisan will understand that gravity feed methods may be used to differentially deliver fluids from two or more chambers, e.g., by varying the head height of each chamber.

The term “manifold” as used herein refers to a discrete structure that receives fluid flow from a plurality of input ports, and allows a resulting flow through a reduced number of output ports. In preferred embodiments, a manifold receives flow from at least three input ports, and allows a resulting flow through a single output port. In particularly preferred embodiments, a manifold receives flow from each non-fluidly connected chamber through a corresponding number of input ports, and allows a resulting flow through a single output port. Flow from one or more input ports to an output port in a manifold may be passive, or may be controlled by one or more valves.

The term “upstream” as used herein refers to any point in a flow path that is closer to the source of the flow path than to the destination of the flow path. An upstream point may also be referred to as a “proximal” location. Similarly, “downstream” refers to any point in a flow path that is closer to the destination of the flow path than to the source of the flow path. A downstream point may also be referred to as a “distal” location.

The phrase “control device” as used herein refers to any device that can reversibly modulate flow down a particular flow path. Control devices of the present invention may be active or passive, as described below, or may serve both an active or passive control function.

The term “valve” as used herein refers to any device within a flow path that starts, stops, or modulates flow through the flow path. Suitable valve configurations are well known to those of skill in the art, including umbrella valves, disc valves, poppet valves, duckbill valves, ball valves, and flapper valves, shuttle valves, gate valves, slit membrane, check valves, and the like.

The phrase “active control device” as used herein refers to any device that can reversibly modulate flow down a particular flow path, and which is not actuated by only the flow or pressure within the flow path itself. An active control device can be one intended to be operated manually, such as a manual valve, stopcock or a pinch clamp, or can be a valve or stopcock that is operated pneumatically, hydraulically, mechanically, by vacuum, or electrically for example. Active control devices may be located within the integral container itself, or along a flow path (e.g., within or along a conduit) between a chamber and the patient. In preferred embodiments, an active control device can reversibly halt all flow down a particular flow path.

The phrase “passive control device” as used herein refers to any device that can reversibly modulate flow down a particular flow path, and which is actuated by only the flow or pressure within the flow path itself. A passive control device can be a valve or stopcock that is opened or closed by altering the flow rate or pressure at the passive control device location. Passive control devices may also be located within the integral container itself, or along a flow path (e.g., within or along a conduit) between a chamber and the patient. In preferred embodiments, a passive control device can reversibly halt all flow down a particular flow path.

As discussed herein, an integral container can be formed from any material from which a container comprising a plurality of integral, non-fluidly connected chambers may be fabricated. As will be appreciated by those of skill in the art, any suitable biocompatible material may be employed in the construction of the integral container, however, it is presently preferred that at least one side of the integral container be transparent to facilitate viewing of the contents. It is also presently preferred that the container be formed of two sheets of flexible material (athough three, four, or more sheets may also be used). For example, the flexible sheets may be ethyl vinyl acetate (EVA), polyvinyl chloride (PVC), polyolefin, or other suitable material. In one embodiment, the first sheet of flexible material has a relatively smooth inner surface and the second sheet of plastic has a texture, such as a taffeta texture (e.g., a diamond taffeta), ribs, or the like, embossed on its inner surface. Alternatively, both sheets may have a patterned inner surface, e.g., a raised diamond taffeta. The sheets are joined together around the perimeter of the container by any means suitable for forming an air and fluid-tight seal that can withstand the pressure generated by the pump apparatus. Fluid-tight seals are also formed between the individual chambers, and should have the same minimum pressure tolerances as the perimeter seals. Thus, the sheets are bonded together to create the patterns for the chambers, conduits, and ports. The materials may be bonded in a variety of ways, e.g., by a radio frequency (rf) seal, a sonication seal, a heat seal, adhesive, or the like, to form an air and fluid-tight seal as described herein.

Each chamber of the integral container preferably has one or more associated conduits. The conduits provide a pathway for fluid to enter and/or exit each chamber. The conduits can be integrally formed during construction of the container, for example, by leaving channels unbonded when the two sheets are fused together to form the container. Optionally, additional internal structure (e.g., rigid or semi-rigid tubing, or the like) may be provided to facilitate fluid flow to and from each chamber.

In an integral container in which fluid is to be delivered by compression of one or more chambers, it is preferred that the conduit through which fluid exits such a chamber lies outside of the compression region (i.e., the region to which pressure is directly applied by contact with a pressure applying structure in the pump apparatus). In this manner, mixing of residual medications in the conduits with subsequently administered medications from other chambers can be minimized. Alternatively, the conduits may lie within the compression region, particularly if mixing is not a concern.

If the conduits are constructed by leaving unbonded channels in the integral container, the conduit will have a generally flat shape but enlarges to have a more tubular shape upon the application of pressure to the corresponding chamber. The shape of the conduit depends on the strength of the materials used to construct the integral container and the pressure of the fluid therein. Specifically, less flexible material (e.g., more rigid or thicker materials) may be more difficult to flex and thus require greater pressure for enlarging the conduit. Advantageously, the textured inner surface of at least one side of the integral container provides flow channels that allow liquid pressure to act along the length of the conduit to assist in opening the conduit upon the application of pressure to the respective chamber. Otherwise, if both inner sides of the container are smooth, surface tension may hold them together and a greater amount of pressure may be required to open the conduits and initiate flow.

The skilled artisan will also understand that a variety of methods may be provided to provide sequential flow control from the various non-fluidly connected chambers within an integral container. One method of providing such control is to configure the integral container itself to provide such sequential flow. Methods and compositions for providing such integral containers are described in U.S. Pat. No. 6,146,360; U.S. Pat. No. 6,074,366; U.S. Pat. No. 6,669,668; U.S. Pat. No. 6,726,555; U.S. patent application Ser. No. 09/713,521, each of which is incorporated by reference herein in its entirety, including all tables, figures, and claims.

In one embodiment of the present invention, the chambers and corresponding conduits from each chamber are arranged in the integral container so that when pressure is applied sequentially from one end of the integral container to the opposite end, individual chambers are sequentially activated. It is presently preferred that the pressure be applied evenly. Even, sequential application of pressure can be accomplished by employing a constant force spring, a roller attached to a constant force spring, a motor-driven roller, or the like.

Additionally, sequential flow control from the various non-fluidly connected chambers within an integral container can be provided by inclusion of one or more pumps, or other means for generating positive or negative pressures, along one or more flow paths between the integral container and the patient. For example, in embodiments where positive pressure is generated, each chamber may be connected to an independently controllable source of pressurization, such as a compressed gas source or a pressurization pump. Similarly, an independently controllable source of negative pressure (e.g., individual pumps or one or more multichannel pumps) can be placed along the flow path from the non-fluidly connected chambers. Suitable pumps are well known in the art. See, e.g., U.S. Pat. Nos. 6,669,668; 6,270,478; 6,213,738; 5,743,878; 5,665,070; 5,522,798; and 5,171,301, each of which is hereby incorporated by reference in its entirety, including all tables, figures, and claims. Pumps useful in the present invention can be simple pumps which are either “on” or “off,” or may comprise a programmable controller (referred to herein as a “smart pump”) that may be integral to the pump or exist as a separate controller unit interfaced in a wired (e.g., via hard wiring, a serial port (such as a standard RS-232 port), a USB port, a “fire wire” port, etc.) or wireless fashion (e.g., connected via an infrared connection, a radio frequency connection, a “bluetooth” connection, etc.).

Suitable programmable pumps are available that permit the operator to generate a pre-defined or user-defined pumping profile. Such pumps may be used to define a volume and rate for fluid flow from each chamber in the integral container. For example, in an integral container having 4 chambers of 10 mL, 100 mL, 10 mL, and 5 mL, the pump could be programmed to run at 1000 ml/hr for the volume of chamber 1, then 200 ml/hr for the volume of chamber 2, then 1000 ml/hr for the volume of Chamber 3, and finally 1000 ml/hr for the volume of chamber 4. Alternatively, four separate pumps (or the individual pumping heads of a 4-channel pump) could be individually or collectively programmed to perform this profile.

Similarly, a pump could be configured to determine a suitable rate, limited by a maximum rate threshold and maximum pressure threshold. In this embodiment, the pump would ramp up the pumping rate until some pre-set maximum rate or pressure was reached. Alternatively, a pump could deliver a “pulsatile” rate, alternating between a preset minimum and a preset or pump-determined maximum rate. These examples are not limiting, and additional pumping profiles could be readily determined by the skilled artisan.

As an alternative to, or in conjunction with one or more pumps for providing sequential flow, one or more active control devices can also be located along one or more flow paths between the integral container and the patient. In these embodiments, controlled actuation of the active control device(s) can provide the required flow control of fluids from the integral container. An active control device can be as simple as a manual pinch valve, which the operator will open as required by the sequential delivery method, or can be a more complicated electrically or pneumatically operated valve. In the latter case, the active control device can be integrally controlled, or can be connected to a controller unit in a wired fashion (e.g., via hard wiring, a serial port (such as a standard RS-232 port), a USB port, a “fire wire” port, etc.) or in a wireless fashion (e.g., connected via an infrared connection, a radio frequency connection, a “bluetooth” connection, etc.).

The various flow paths (ports, conduits, etc.) from each non-fluidly connected chamber to the patient will preferably merge at some point into a single conduit through which fluids are infused to the patient. Numerous methods are well known to the skilled artisan to merge such flow paths. These can include simple connections, such as 3-way (or 4-way, or 5-way, etc.) connectors in which two (or three, or four, etc.) input paths flow out through a single output path. In more complex arrangements, the placement of valves (arranged in parallel or series, or a combination of the two) on each flow path, and/or one or more manifold units can provide the required merger of flow paths. Exemplary manifolds are described hereinafter. Other manifolds are disclosed in, e.g., U.S. Pat. Nos. 5,374,248; 5,217,432; and 5,431,185, each of which is hereby incorporated by reference in its entirety, including all tables, figures, and claims. Manifolds may additionally contain one or more control devices, either active, passive, or a combination thereof, to control the flow of fluid through the manifold.

In embodiments where negative pressure is used to withdraw fluid from the chambers of an integral container, the skilled artisan will understand that the overall configuration of the device may depend on the position of the pump relative to the merger of the various flow paths. For example, a plurality of pumps (or a multichannel pump) corresponding to a plurality of flow paths flowing into a manifold may be placed upstream from the manifold, thereby providing a means to provide negative pressure along each flow path. Alternatively, a single pump placed downstream of a manifold may be used to provide negative pressure along each flow path that flows into the manifold.

It may be desirable to include an accumulator chamber, either in the integral container itself or on one or more flow paths leading from the container. Some positive displacement pumps have a relatively high flow rate when the displacement chamber in the pump is being refilled. If the refill flow rate is faster than the gravity flow rate from the multi-chambered container, fluid could potentially be pulled out of more than one chamber. To avoid this, an accumulator chamber that is preferably flexible, is placed in series with the pump. Fluid may be permitted to flow (e.g., by gravity) into the accumulator until it is full, and when the downstream pump pulls fluid, it will pull only from the accumulator. Flow out of the appropriate chamber will then re-fill the accumulator for the next fill stroke of the down stream pump.

It may also be desirable to mix the contents of two or more chambers immediately prior to administration to form a single chamber that is not fluidly connected to other chambers within the integral container. Accordingly, in another embodiment of the present invention, frangible seals between two or more adjacent chambers may be formed. In this manner, upon application of pressure sufficient to rupture the seal, the contents of selected adjacent chambers will be mixed. The chambers may be side by side, parallel or perpendicular relative to one axis of the integral container.

Chambers may also be configured to have a “blow down” period between activation of one chamber and activation of the next chamber during an infusion sequence to prevent mixing of medications during the infusion. As described in greater detail below, this can be accomplished, for example, by providing a space between adjacent chambers, or the like.

In those embodiments where the integral container is of a flexible configuration (e.g., a bag) it has been observed that there can be a pressure drop between a chamber and its corresponding conduit when pressure is applied to the contents of the integral container. This is largely due to the formation of kinks in the flexible material when pressure is applied to the contents of the integral container. The region of primary concern is the interface between the chamber and its corresponding conduit. Thus in one embodiment of the present invention, structure is provided to alleviate pressure drop between each chamber and its corresponding conduit. This can be achieved by one or more of several methods, including quilting of the chamber, incorporation into the chamber of internal structures (e.g., a stent, tubing, conduit bead(s), solid filament, or the like), employing external structures (e.g., a source of pressure on the container, such as a protruding member of the pump apparatus, or the like), and the like. Additionally, the width, angle and/or taper of the conduit, the thickness of the chamber or conduit, and/or the type of material forming the chamber or conduit may be selected to minimize flow resistance.

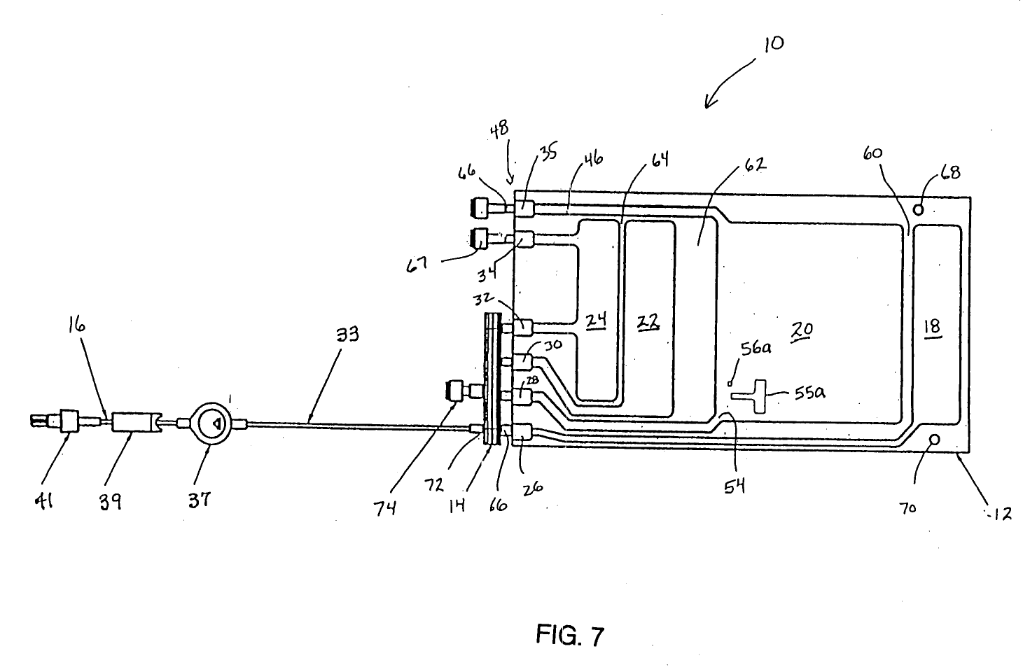

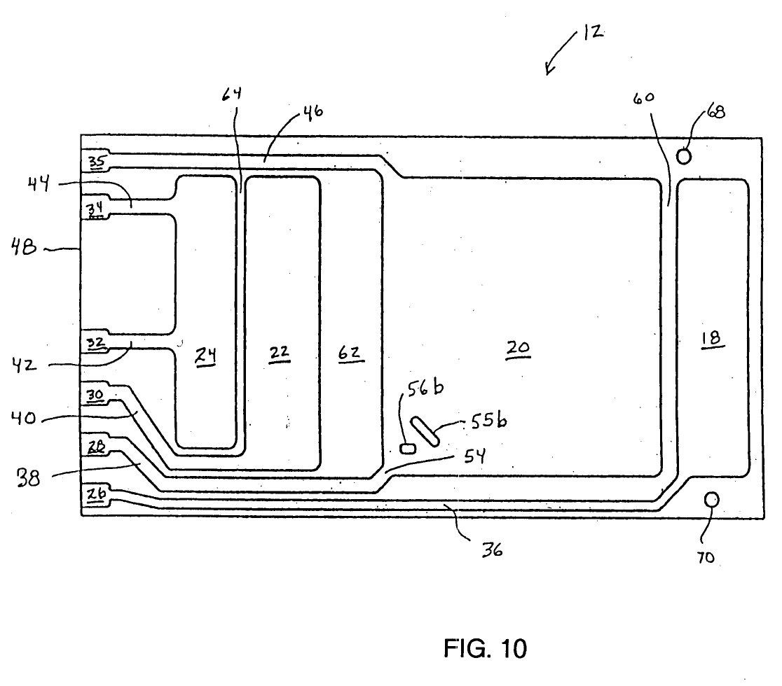

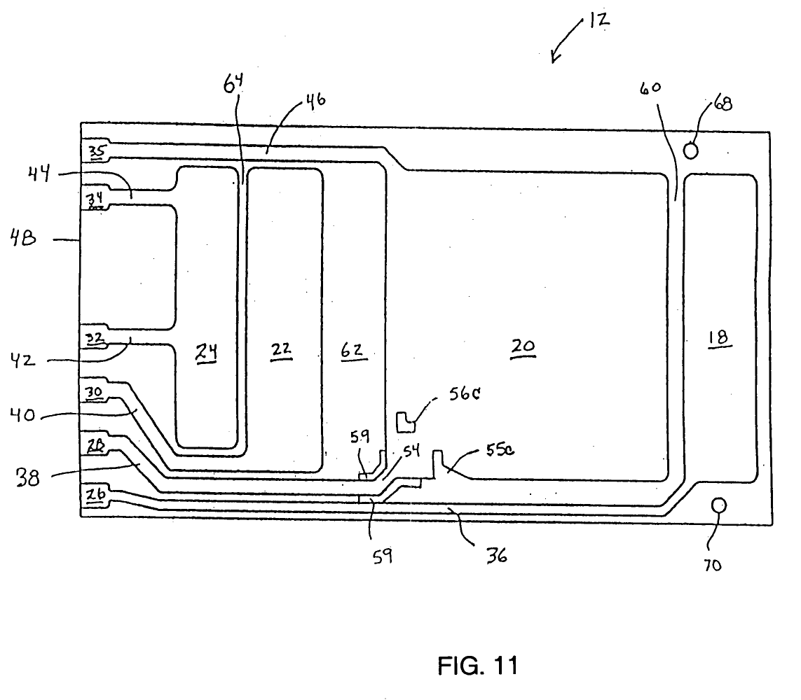

As used herein, “quilting” means forming a structure in the interior of the chamber wherein the bottom and top sides of the integral container are connected, preferably by fusing them together. It is presently preferred that quilting be employed to manage pressure drop, as the desired connection between first and second sides of the integral container can be accomplished by the same methods used to form the perimeter seal of the container. Quilting may be at any region of the chamber that provides a substantially reduced or eliminated pressure drop between the chamber and its corresponding conduit. It is presently preferred that the quilting be in the region of the chamber that is proximal to the conduit. In this region of the chamber, any one of a number of quilt shapes may be employed, including a T dot configuration, 55a and 56a, as shown in FIG. 7, a dash dot configuration, 55b and 56b, as shown in FIG. 10, bond blocks 55c and 56c, as shown in FIG. 11, or the like. These types of quilting are discussed in greater detail below in reference to specific embodiments.

Other features suitable for minimizing flow resistance (i.e., pressure drop) caused by kinks include thermoforming of the conduit, introduction of an internal conduit bead in the region where the conduit joins the chamber, coining, or the like. Thermoforming involves heating the integral container materials in the region of the exit and associated conduit until the materials are softened slightly. Air pressure is applied to the chamber to open (or inflate) the exit and the conduit. The material is allowed to cool such that the exit and conduit retain a slightly circular opening or cross-section after the pressure is removed. In certain embodiments, a mold may be used to constrain the shape of the blow-molded conduit. For employing internal conduit bead(s), a portion of the bag adjacent the exit to the conduit is stamped with an offset bonding pattern or shim to provide a three-dimensional structure in the region of the exit. (See, e.g., structure 59, FIG. 11). This can be analogized to gluing two sheets of paper together at their perimeter and affixing a solid piece, like a bamboo skewer, along the length of the seam between the two sheets. In this manner, even when the two sheets are pressed together, a channel will exist along the skewer where the sheets are prevented from meeting one another. Additionally, coining (i.e., forming a structured pattern in the integral container material) may be applied to the sides of the integral container in the region of the exit to provide additional flow pathways not subject to greatly restricted flow by kinks.

It is contemplated that each conduit will have an associated port where, at a minimum, fluids exit the integral container. These conduits may serve the dual purpose of providing a channel for both the introduction of fluids into the chamber(s) and exit of fluids from the chambers. The container may have one or more ports for introduction of fluids into one or more of the individual chambers of the container. In one embodiment, these ports have associated conduits, separate from the exit conduits. The ports are configured to allow regulated, sterile introduction of fluids. This can be accomplished by fitting the ports with injection ports, or the like.

Containers may be filled in a variety of ways by suitable personnel, e.g., by a pharmacist. Similarly, the container may be provided to a pharmacist in a variety of states. For example, the bag may be provided filled, or empty for subsequent sterilization and filling at the pharmacy. It is presently preferred that the multi-chambered bag is provided sterile and is then filled at the pharmacy. The bag may be appropriately filled using standard pharmaceutical admixture procedures and equipment. Each chamber may be manually filled using injection ports or the like. Alternatively each chamber can be filled by introduction of fluids into a common filling conduit that branches off to the respective fill or dual purpose fill/exit conduit associated with each chamber. Once the bag is prepared, it is labeled and sent from the pharmacy to the end user.

As discussed above, the fluid delivery devices of the present invention can comprise a manifold to regulate delivery of fluids from the ports on the integral container corresponding to each chamber to an administration tube set (“administration set”). Such a manifold may optionally provide a structure for filling one or more chambers. As used herein, “container port of the conduit” and “container port” refer to the terminal portion of each conduit leading to/from a chamber in the integral container. The container ports may have an adapter affixed thereto for mating the ports with the manifold, or the manifold may be attached directly to the container ports. The manifold can be any structure that is attachable to the bag ports (or adapters) in a fluid-tight manner while providing a common outlet for all bag ports to the administration set.

In describing the manifold, reference will be made to the “integral container side” of the manifold (where the manifold attaches to the integral container ports) and the “infusion side” (where the manifold attaches to the administration set). Further reference will be made to chamber ports of the manifold, where the manifold attaches to and is in fluid communication with the chamber ports. Additional reference will be made to an output port of the manifold, where the manifold attaches to and is in fluid communication with the administration set. Although optional, it is presently preferred that the manifold also have a bulk fill port, where the manifold can be attached to, and be in fluid communication with, a source of fluids for introduction into the integral container.

Manifolds contemplated for use in the practice of the present invention will have manifold conduits for directing fluid from chamber ports to the output port for exit to the administration set, and from the bulk fill port, when employed, to the chamber ports. These manifold conduits can be isolated from one another in a fluid-tight manner and can comprise internal chambers connecting the desired portions of the manifold, or they may comprise internally mounted tubing connecting the appropriate portions of the manifold, combinations thereof, or the like.

In order to regulate the flow of fluid through the manifold and to prevent backflow from the output port to the chamber ports, it is presently preferred that the manifold have check valves therein. Check valves can be configured in a variety of manners to regulate fluid flow as desired; all such configurations are contemplated as being within the scope of the present invention. In one embodiment of the present invention, fluid flow is regulated so that fluid exiting the container and entering the manifold through the chamber ports can only exit the manifold through the output port without returning to the bag by way of any other chamber port. This is accomplished by interposing a first check valve in a first conduit between each chamber port and the output port. The check valve only allows fluid to flow from the bag side of the manifold towards the infusion side where the output port is located.

It is important to note that some or all of the chambers may be individually filled by way of optional separate fill ports on the integral container and/or by way of the optional bulk fill port of the manifold. In an embodiment of the present invention, when a bulk fill port is to be used, fluid flow in the manifold is further regulated so that fluid introduced through the bulk fill port can access one or more of the chamber ports for filling of chambers in the integral container. Accordingly, chamber ports to be used for both filling and dispensing fluids will have two manifold conduits associated therewith: a first manifold conduit, as described above, for directing fluids from the chamber port(s) to the output port; and a second manifold conduit branching off of the first at a point between each chamber port and the first check valve. In this embodiment, a second check valve is located on each second manifold conduit between the chamber port and the bulk fill port. The second check valve only allows fluid to flow from the bulk fill port towards the chamber port. A schematic of one example of this embodiment is provided in FIG. 15, as further described below.

Any type of check valve can be employed in the practice of the present invention, including ball check valves, umbrella check valves, and the like. In a presently preferred embodiment of the present invention, an umbrella check valve is employed. Umbrella valves are inexpensive, simple in their operation and easy to install. Because umbrella valves are held in place by friction, it is presently preferred that the interior of the manifold be configured so that, upon assembly of the manifold, the umbrella valves are held securely in place by the internal structure of the manifold. This can be accomplished simply by having a structure that contacts the center of the umbrella portion (i.e., the dome of the umbrella) to bias the valve towards its associated passageway. In this manner, the force of liquid flowing past the valve will open but not unseat the valve.

The ports, valves and conduits of the manifold may be configured in any manner that permits the desired flow of fluid through the manifold. It is presently preferred that the conduits and output port be configured so that fluid exiting each sequentially activated bag chamber flows through its associated first check valve and then past all conduits leading from previously emptied bag chambers, before the output port is encountered. In this manner, residual fluid output from each bag chamber is pushed through the manifold and out through the output port by fluid from subsequently emptied bag chambers.

In order for the fluid flow to be further regulated (e.g., to prevent unintentional fluid flow from the bag through to the output port), it is desirable that the check valves be controllable as to when flow is permitted therethrough. This can be accomplished in a number of ways, depending on the type of check valve employed. For example, a valve can be employed having a threshold operating pressure (i.e., a cracking pressure) that opens the valve. The cracking pressure of the valve may be any pressure suitable for the intended application. Suitable cracking pressures should be no higher than the pressure generated by the pump apparatus, yet high enough to prevent unintentional flow through the manifold. Preferred cracking pressures can be in the range of about 0.25 lbs per square inch up to about 2 lbs per square inch. It is more preferred that the cracking pressures be in the range of about 0.50 lbs per square inch up to about 1 lbs per square inch. In a most preferred embodiment, the cracking pressure is about 0.75 lbs per square inch. The cracking pressures should be consistent in a given direction of fluid flow. Thus, the check valves associated with the chamber ports and the output port can have one cracking pressure while the check valve(s) associated with the bulk fill port has a different cracking pressure. Due to economies of scale, it is presently preferred that the valve types and cracking pressures be consistent throughout the manifold.

An administration set is optionally provided in one embodiment of the present invention. The administration set comprises a length of medical grade tubing, such as a micro-bore tube, or the like, with structures at each end: at one end (proximal end) for connecting the tubing to the output port of the manifold and at the opposite (distal) end for connection to a standard intravenous-type needle. Standard luer connectors, needleless connectors, or the like may be used in the practice of the present invention.

The administration set may be further configured to regulate the rate of fluid administration to the patient. It is necessary to know the pressure generated by the pump/manifold combination in order to calibrate the delivery rate of the administration set. The devices of the present invention may be configured so that gravity or the pump apparatus generates predictable fluid pressures based on the volume of solution in each chamber. Using the predictable fluid pressures, the flow rate from the integral container may be selectable using administration sets having predetermined tubing lengths and inner diameters. The flow rate through the administration set is selected by varying the microbore tubing's inner diameter and length. The relationship is approximated by Poiseulle's equation:
Error! Objects cannot be created from editing field codes. Equation 1
where Q is the flow rate, Δp is the pressure drop across a flow controlling orifice, D is the inside diameter of the orifice, μ is the dynamic viscosity of the fluid and L is the length of the orifice. Thus, any structures included in the administration set will effect the flow rate in a predictable and calculable manner. Structures contemplated for optional incorporation into the administration set include particulate filters, air elimination filters, fluid flow restrictors, flow indicators, drop counters, drip chambers, pressure indicators, and the like. The administration set may further comprise a clamp, or the like, for stopping fluid flow, as desired.

In another embodiment of the present invention there is provided a restrictor set for attachment to the distal end of the administration set. In this manner, the rate of fluid flow can be altered with the simple addition of a restrictor set, rather than by re-engineering the administration set. Of course, the maximum fluid flow rate will be determined by the configuration of the administration set, with fine-tuning to slower rates provided by the restrictor set. Restrictor sets may be located at a variety of positions in a flow path, such as in a chamber, in a conduit, in a manifold, etc.

The integral containers of the present invention may be provided to a user (e.g., a physician or pharmacist) in an unfilled state for subsequent filling with fluids deemed appropriate by the user; that is, the bags may be configured by a clinician or pharmacist to deliver a regimen of fluids deemed advantageous to a particular patient. Alternatively, one or more, and preferably all, chambers within the integral container may be provided to the user pre-filled with fluids to be delivered in a predetermined sequence.

The integral containers and methods described herein can provide a methodology by which a course of therapy involving multiple fluids can be preconfigured and stored, e.g., in a hospital or pharmacy, for “off the shelf” delivery to the clinician or patient. Additionally, the integral containers and methods described herein allow for the careful preselection of fluids, to ensure that none of the fluids to be delivered to a patient from the integral container will interact adversely with other fluids to be delivered from the same integral container. The present invention contemplates that any compounds or groups of compounds that may be delivered in a fluid format may be delivered to a patient in accordance with the foregoing description. An exemplary list of suitable compounds is provided below.
analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like);
antimigraine agents (e.g., ergotamine tartrate, propanolol hydrochloride, isometheptene mucate, dichloralphenazone, and the like);
sedatives/hypnotics (e.g., barbiturates (e.g., pentobarbital, pentobarbital sodium, secobarbital sodium), benzodiazapines (e.g., flurazepam hydrochloride, triazolam, tomazeparm, midazolam hydrochloride, and the like);
antianginal agents (e.g., beta-adrenergic blockers, calcium channel blockers (e.g., nifedipine, diltiazem hydrochloride, and the like), nitrates (e.g., nitroglycerin, isosorbide dinitrate, pentaerythritol tetranitrate, erythrityl tetranitrate, and the like));
antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlornezanone, and the like);
antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine, and the like);
antimanic agents (e.g., lithium carbonate),
antiarrhythmics (e.g., bretylium tosylate, esmolol hydrochloride, verapamil hydrochloride, amiodarone, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, lidocaine hydrochloride, and the like);
antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium, and the like);
antigout agents (e.g., colchicine, allopurinol, and the like);
anticoagulants (e.g., heparin, heparin sodium, warfarin sodium, and the like);
thrombolytic agents (e.g., urokinase, streptokinase, altoplase, and the like);
antifibrinolytic agents (e.g., aminocaproic acid);
hemorheologic agents (e.g., pentoxifylline);
antiplatelet agents (e.g., aspirin, empirin, ascriptin, and the like);
anticonvulsants (e.g., valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, trimethadione, and the like);
antiparkinson agents (e.g., ethosuximide, and the like);
antidepressants (e.g., doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, protriptyline hydrochloride, and the like);
antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like);
antihypertensive agents (e.g., trinmethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine, and the like);
agents useful for calcium regulation (e.g., calcitonin, parathyroid hormone, and the like);
antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate, and the like);
antifungal agents (e.g., griseofulvin, keloconazole, and the like);
antiviral agents (e.g., interferon gamma, zidovudine, amantadine hydrochloride, ribavirin, acyclovir, and the like);
antimicrobials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like), penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, and the like), erythromycins (e.g., erythromycin ethylsuccinate, erythromycin, erythromycin estolate, erythromycin lactobionate, erythromycin siearate, erythromycin ethylsuccinate, and the like), tetracyclines (e.g., tetracycline hydrochloride, doxycycline hyclate, minocycline hydrochloride, and the like), and the like);
anti-infectives (e.g., GM-CSF);
bronchodialators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate), anticholinergic agents (e.g., ipratropium bromide), xanthines (e.g., aminophylline, dyphylline, metaproterenol sulfate, aminophylline), mast cell stabilizers (e.g., cromolyn sodium), inhalant corticosteroids (e.g., flurisolidebeclomethasone dipropionate, beclomethasone dipropionate monohydrate), salbutamol, beclomethasone dipropionate (BDP), ipratropPl. bromide, budesonide, ketotifen. salmeterol, xinafoate, terbutaline sulfate, triamcinolone, theophylline, nedocromil sodium, metaproterenol sulfate, albuterol, flunisolide, and the like);
hormones (e.g., androgens (e.g., danazol, testosterone cypionate, fluoxymesterone, ethyltostosterone, testosterone enanihate, methyltestosterone, fluoxymesterone, testosterone cypionate), estrogens (e.g., estradiol, estropipate, conjugated estrogens), progestins (e.g., methoxyprogesterone acetate, norethindrone acetate), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and the like), thyroid hormones (e.g., levothyroxine sodium) and the like), and the like;
hypoglycemic agents (e.g., human insulin, purified beef insulin, purified pork insulin, glyburide, chlorpropamide, glipizide, tolbutamide, tolazamide, and the like);
hypolipidemic agents (e.g., clofibrate, dextrothyroxine sodium, probucol, lovastatin, niacin, and the like);
proteins (e.g., DNase, alginase, superoxide dismutase, lipase, and the like);
nucleic acids (e.g., sense or anti-sense nucleic acids encoding any protein suitable for delivery by inhalation, including the proteins described herein, and the like);
agents useful for erythropoiesis stimulation (e.g., erythropoietin);
antiulcer/antireflux agents (e.g., famotidine, cimetidine, ranitidine hydrochloride, and the like); and
antinauseants/antiemetics (e.g., meclizine hydrochloride, nabilone, prochlorperazine, dimenhydrinate, promethazine hydrochloride, thiethylperazine, scopolamine, and the like).

This list is not intended to be limiting. Additional agents contemplated for delivery employing the devices and methods described herein include agents useful for the treatment of diabetes (e.g., activin, glucagon, insulin, somatostatin, proinsulin, amylin, and the like), carcinomas (e.g., taxol, interleukin-1, interleukin-2 (especially useful for treatment of renal carcinoma), and the like, as well as leuprolide acetate, LHRH analogs (such as nafarelin acetate), and the like, which are especially useful for the treatment of prostatic carcinoma), endometriosis (e.g., LHRH analogs), uterine contraction (e.g., oxytocin), diuresis (e.g., vasopressin), cystic fibrosis (e.g., Dnase (i.e., deoxyribonuclease), SLPI, and the like), neutropenia (e.g., GCSF), lung cancer (e.g., beta 1-interferon), respiratory disorders (e.g., superoxide dismutase), RDS (e.g., surfactants, optionally including apoproteins), and the like.

Presently preferred indications which can be treated employing the device and methods described herein include diabetes, carcinomas (e.g., prostatic carcinomas), bone disease (via calcium regulation), cystic fibrosis and breathing disorders (employing bronchodilators), and the like.

In accordance with the present invention, there are also provided medication delivery containers that are configured to administer an infusion therapy upon activation by a pump mechanism. The container is preferably further configured to interface with a pump apparatus in a manner that securely maintains the container in position during pumping.

The invention container comprises a multi-chamber bag wherein the chambers, each configured to deliver predetermined amounts of liquid medication at a predetermined rate and pressure, and each placed in relation to the others in a manner that determines the order in which the fluids contained therein, are administered.

Each chamber has an associated exit conduit whereby fluid can exit each chamber for administration to a patient. Thus, for example, a container might have four separate chambers, each sized to hold a different amount of fluid. The container can be filled so that each chamber has a different medication therein. If the four chambers are arranged sequentially in the bag from one end of the bag to the other, and each chamber is activated sequentially from one end of the bag to the other, then fluid will be driven out of the first chamber, and then the second, and so on until each chamber has been emptied.

Each chamber has one or more associated conduits. The conduits provide a pathway for fluid to enter and/or exit each chamber. The conduits can be integrally formed during construction of the container, for example, by leaving channels unbonded when two flexible sheets are fused together to form the container. Optionally, additional internal structure (e.g., rigid or semi-rigid tubing, or the like) may be provided to facilitate fluid flow to and from each chamber. It is presently preferred that the conduits through which medication exits the chambers lie outside of the compression region (i.e., the region to which pressure is directly applied by contact with a pressure applying structure in the pump apparatus). In this manner, mixing of residual medications in the conduits with subsequently administered medications from other chambers is minimized. Alternatively, the conduits may lie within the compression region, particularly if mixing is not a concern.

Because the container is to be subject to the sequential application of pressure, it is desirable for the container to be anchored inside the pump apparatus in a manner that prevents the pressure application device from merely moving the container ahead of it as the pressure is applied from one end of the bag to the other. Accordingly, it is presently preferred that the container be anchorable to the pump apparatus. This can be accomplished in a variety of ways, including the use of fasteners secured to the bag that will mate with counterpart fasteners in the pump apparatus. Such fasteners include hook and loop fasteners, snaps, buttons, zippers, and the like. In a presently preferred embodiment, the container is anchored by forming holes in a non-fluid containing portion of the bag, and mating these holes with corresponding protrusions such as pins, or the like, in the pump housing. These anchoring structures can serve the dual purpose of securing the bag and positioning it properly in the pump apparatus. This latter purpose can be accomplished by orienting the attachment structures so that there is only one orientation with which the bag can be positioned in the pump apparatus.

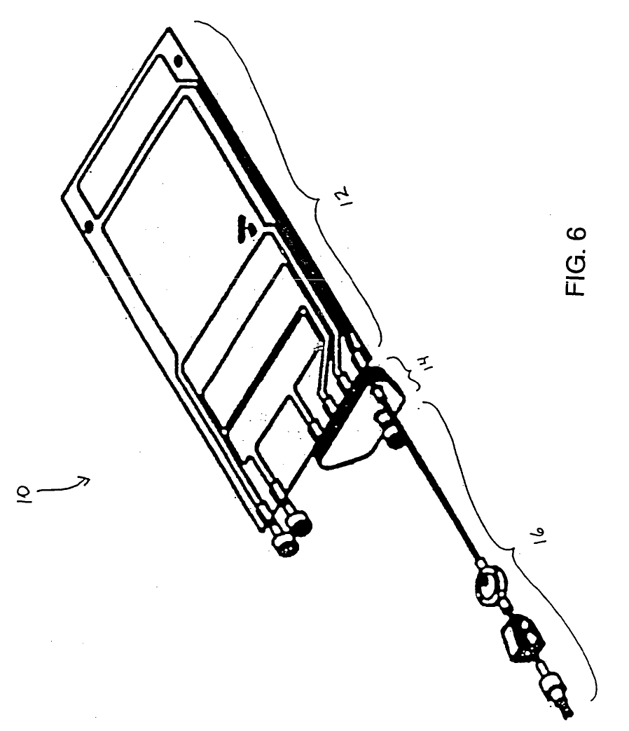

With reference to FIGS. 6 and 7, the medication delivery container 10 of the invention includes a multi-chamber bag 12, a manifold assembly 14 and a tube assembly 16. The container provides improved infusion therapy administration which is particularly advantageous for reducing errors, infections and other complications associated with manual infusion techniques.

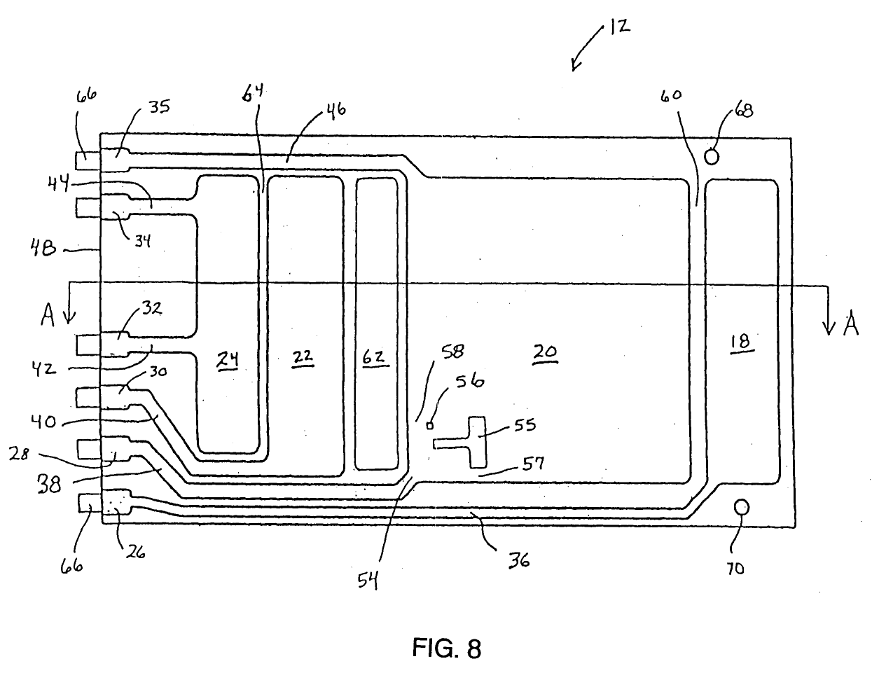

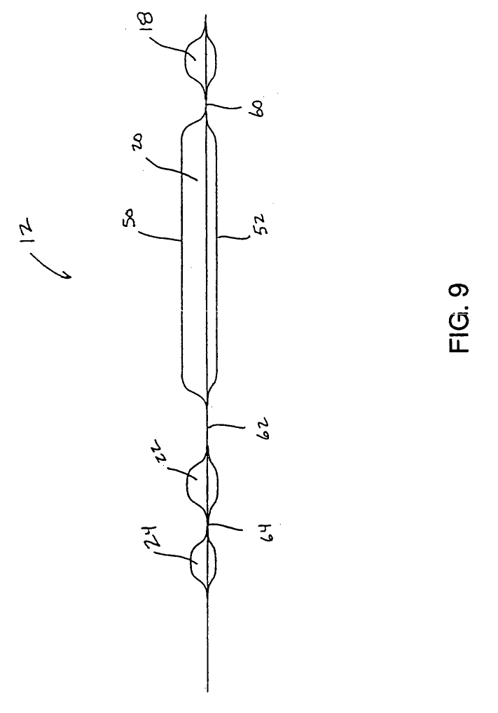

The multi-chamber bag, as shown in FIGS. 8 and 9, may include four chambers 18, 20, 22 and 24, six ports 26, 28, 30, 32, 34 and 35, and six conduits 36, 38, 40, 42, 44 and 46 for coupling each of the respective ports to a chamber. The multi-chamber bag may have other chamber, port and conduit configurations of varying number, sizes, and shapes in accordance with the invention. The ports may lie at the end 48 or along one or more edges of the bag. The chambers comprise a relatively large area of the bag in a central portion of the bag and are configured to be filled with medication fluids or pharmacological agents. The central chamber portion of the bag may be referred to as a compression region which is sequentially compressed by application of an external pressure (e.g. a pump having a constant force spring as described herein) to drive liquid from the chambers through the respective conduits and out the ports in accordance with the infusion therapy. The conduits generally lie outside of the compression region to avoid residual medications in the conduits from mixing with subsequently administered medications from other chambers. The conduits may lie within the compression region particularly if mixing is not a concern.

The multi-chamber bag 12 is preferably formed of two flexible sheets 50 and 52, of material and has a generally rectangular flat shape. The flexible sheets may be ethyl vinyl acetate (EVA), polyvinyl chloride (PVC), polyolefin or other suitable material. One sheet may have a relatively smooth inner surface and the other sheet may have a taffeta texture (or similar pattern that is not smooth, such as ribs) embossed on its inner surface. Alternatively, both sheets may have an inner surface that is not smooth. The sheets are bonded together to create the patterns for the chambers, conduits, and ports. The materials may be bonded by suitable means, e.g., by a radio frequency (rf seal, sonication, by heat seal, adhesive, or the like, to form an air and fluid tight seal between the chambers and the conduits. When filled with medication fluids, the chambers bulge creating a “pillow-like” shape (FIG. 9). It is also presently preferred that at least one side of the bag be transparent to facilitate viewing of the contents.

The first chamber 18 is furthest from the port side 48 of the bag and may contain a first medication fluid of an infusion therapy sequence. The first chamber is coupled to a first bag port 26 by a first conduit 36. The first chamber is filled with fluid through the first bag port.

The spacings 60, 62 and 64 between the chambers advantageously provides a “blow-down” period during an infusion sequence to prevent mixing of medications during the infusion. The spacing 62 between the second chamber 20 and the third chamber 22 is sized based on the time needed for the chamber and conduit to “blow down”, or flow until the residual pressure is below the cracking pressure of the associated check valves in the manifold. The area of the spacing 62 may be sealed only around the perimeter with no bond between completion of the sheets in the central spacing area to provide additional kink and flex absorbing characteristics to the bag. This spacing 62 is configured to allow a sufficient time period between completion of the infusion of the medication in the second chamber and the beginning of the infusion of medication in the third chamber so as to minimize or prevent mixing of the medication in the second chamber with the medication in the third chamber. This time period is sufficient to allow the material spring strength of the flexible sheets, 50 and 52, that form the conduits to pull the respective conduit 38 flat to expel residual fluid from the conduit. The time required will, of course, vary with the size of the chamber, the rate of infusion, and the like. Note that the spacing 60 between the first chamber 18 and the second chamber is effectively as large as the spacing 62 because a significant portion of the second chamber must be compressed before the pressure is sufficient to expel residual fluid from the second chamber. Thus, the spacing between chambers provides a delay between chambers to allow expulsion of residual conduit fluid before the start of the infusion of medication from the next chamber. This is especially advantageous for preventing mixing of agents from non-adjacent chambers.

The second chamber 20 typically has the largest fluid volume of the four chambers. As discussed in more detail below, the second chamber is coupled to the second port 28 and the sixth port 35 by respective conduits. When filled with medication, the second chamber has a pillow-like shape. As a result of the relatively large pillow-like shape of the second chamber (and the flexible nature of the materials used to construct the bag), when pressure is applied to the second chamber, there may be a resistance to flow because the chamber has a tendency to kink near the chamber exit 54 to the conduit, often cutting off fluid flow to the conduit. To prevent a pressure drop due to kinks from forming at the exit port, a “quilt” pattern of bonds may be placed near the exit. The quilt pattern may consist of two spot bonds, 55a and 56a, having a “T dot” configuration. The quilt pattern moves the chamber's kinking tendencies to other areas of the bag where kinking is not of concern, away from the exit 54. The first bond 55a has a “T” shape providing first and second openings, 57 and 58. From observation, it appears that the cross bar of the T causes the chamber to kink laterally and preferentially above the outlet 54. The leg of the T further causes a longitudinal kink away from the outlet 54. After the chamber has been compressed to the first opening 57, the “pillow” of the compressed chamber is of a size that is less susceptible to exit kinks. The second “dot” bond further discourages kinking of the second opening 58. The quilt pattern may be provided to other ports of the chamber to prevent kinking while removing air, etc. Empirical tests have determined that the quilt pattern configuration discourages kinks at the exit and allows reliable delivery of the medication from the second chamber into the respective conduit 38.

In an alternative embodiment of the invention, the quilt pattern may consist of the two spot bonds, 55b and 56b, shown in FIG. 10. The first spot bond 55b may have a generally elongated oval shape and may be preferably placed at a 45 degree angle with respect to the chamber sides. The second spot bond 56b may have a shorter oval shape and is preferably placed between the first spot bond and the exit or entrance to conduit 38.

In another embodiment of the invention, the quilt pattern may consist of the bond blocks, 55c and 56c, shown in FIG. 11. The first bond block may have a generally elongated angle shape with a protrusion and may be preferably placed about ½ inch from the exit 54 to conduit 38. The second bond block may have a corner shape and is preferably placed nearly between the first bond block and the exit 54 (or entrance) to conduit 38.

Referring to FIG. 7, the third chamber 22 is coupled to the third port 30 by a respective conduit 40. The fourth chamber 24 is coupled to the fourth and fifth ports, 32 and 34, by respective conduits 42 and 44.

The six ports are used to fill and/or empty the fluid in the chambers. Two of the ports, the fifth and sixth ports, 34 and 35 (see FIG. 7, for example), are directly coupled to the fourth chamber 24 and second chamber 20, respectively. The four remaining ports, 26, 28, 30 and 32, are coupled to a manifold assembly 14 for filling the chambers and for delivering the medications of the infusion therapy. A short plastic tube 66 couples each respective port to the respective manifold port or injection fill site 67. The tubes extend into the ports between the plastic sheets, 50 and 52, and are sealed to the sheets to form closed, sealed fluid connections.

The bag may be constructed of an EVA (ethylene vinyl acetate) or like film material which is often used in the construction of intravenous solution containers. This material is generally rugged, durable and biocompatible. The bag is configured to withstand pressures greater than those achieved during an infusion. The interior of the pump housing where the bag resides is configured such that a filled bag will be positioned correctly and securely. In the depicted embodiment, this is accomplished by the use of registration pins 151 (or similar features) in the pump receptacle (FIG. 17) to engage, for example, corresponding holes 68 and 70 in the bag (See FIG. 7).

The tubes may be formed of co-extruded plastic for providing a compatible bonding surface. For example, if the bag 12 is formed of EVA and the manifold is formed of acrylonitrile butadiene styrene (ABS), the co-extruded tube 66 would have an exterior of EVA and an interior of PVC. The outside of the tube (FVA) would be heat sealed to the bag (EVA) and the inside of the tube (PVC) would be solvent bonded to the outside of a corresponding port of the manifold (ABS).

In one application of the invention, the first, second and third chambers, 18, 20 and 22, may be filled with a diluent such as a saline solution, a dextrose solution or sterile water, and the fourth chamber 24 is filled with heparinized saline (e.g., through the fifth port 34). A medication, such as an antibiotic, may be injected into the second chamber through the sixth port 35 before commencing delivery of the infusion therapy to a patient.

The multi-chamber bag 12 also may include a plurality of alignment holes, e.g., 68 and 70. The alignment holes may be offset and aligned with corresponding features such as pins in a pump. The alignment holes ensure that the bag is installed into the pump in the correct position, and maintained in that position during pumping.

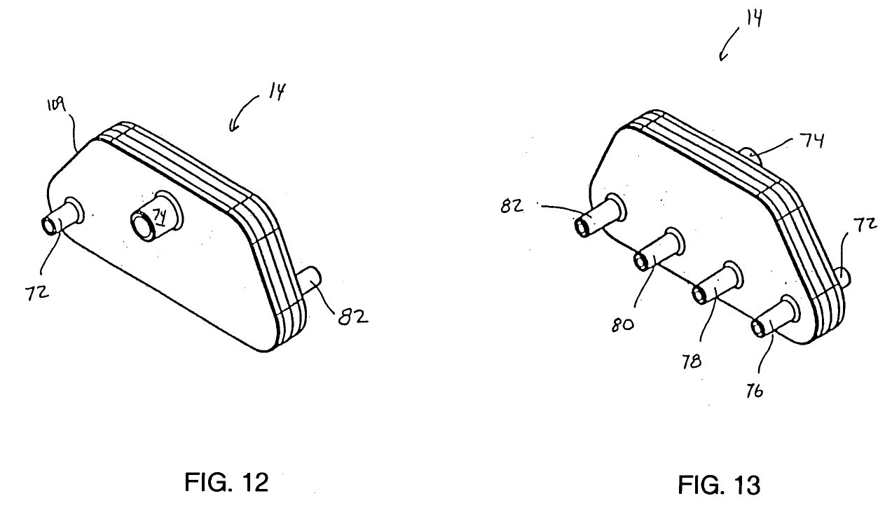

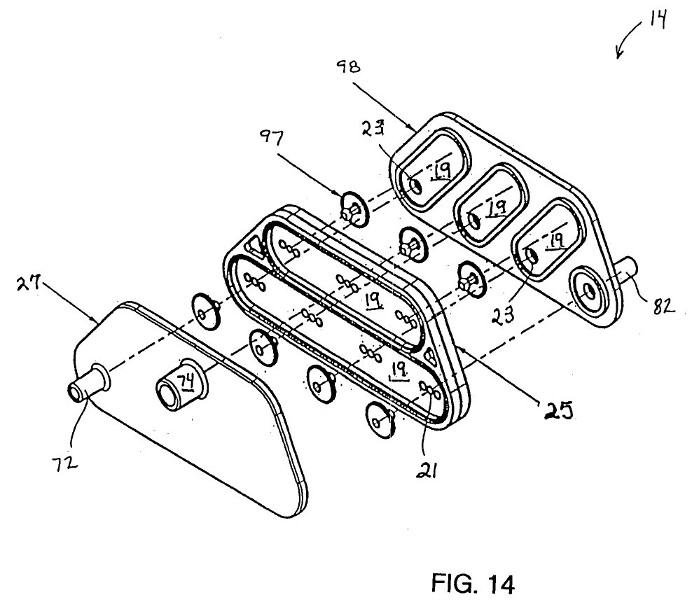

With reference to FIGS. 12-13, the manifold assembly 14 has a tube or output port 72, a bulk fill port 74 and four chamber ports 76, 78, 80 and 82. The four chamber ports are connected, respectively, to the first, second, third and fourth bag ports 26, 28, 30 and 32 (see FIG. 11). The manifold assembly allows filling of the first, second and third chambers, 18, 20 and 22, through the bulk fill port and delivery of the fluids in the first, second, third and fourth chambers through an output port. Seven check valves, 84, 86, 88, 90, 92, 94 and 96 (FIG. 14), control the fluid flow direction within the manifold, in concert with manifold conduits formed by bonding manifold pieces together. The manifold assembly may have additional or fewer check valves and ports based on the number and configuration of chambers implemented by the multi-chamber bag.

In a particular embodiment, as shown in FIG. 14, for example, the manifold assembly may be constructed of three molded pieces and seven check valves. The three molded pieces may be formed of any suitable biologically compatible rigid or semi-rigid material, e.g., ABS plastic, or the like. The three molded pieces are a bag side piece 98, a middle piece 25, and an infusion side piece 27. The bag side piece has the four chamber ports 76, 78, 80 and 82. The bag side piece also has recesses 23 for three of the umbrella valves 97 and conduits 19 for directing fluid flow between the ports in conjunction with the other manifold pieces. The middle piece 25 has valve through holes 21 for receiving the umbrella valves and for providing fluid communication through the middle piece. The middle piece also has conduits on both sides that correspond to the conduits on the respective side pieces. The infusion side piece 27 includes the output port 72 and the bulk fill port 74. The infusion side piece also has internal recesses (not shown) for four of the umbrella valves and conduits (not shown) for directing fluid flow within the manifold assembly, as well as protrusions designed to contact the middle of the dome of the umbrella for biasing the check valve in the proper position. The three manifold pieces are attached together by suitable adhesive, clips or the like to form the manifold assembly. The manifold assembly can be further shaped so that it can only be correctly placed in a corresponding receptacle in the pump apparatus. For example, the manifold may include one or more beveled edges 109 (FIG. 12) for correctly aligning the container 10 in a pump mechanism.

The medication delivery container 10 may have a wide variety of configurations and dimensions based on the prescribed infusion therapy. For example, when infusion therapies permit (e.g., when small volumes of concentrated solution are to be infused), bags may be sufficiently small to incorporate into an easily portable pump apparatus. Chambers may be configured for the simultaneous infusion of medicaments from separate chambers. Empirical evaluation of the container and manifold configuration shown in FIGS. 6-15 has demonstrated effective delivery of fluids.

In accordance with another embodiment of the present invention, there is provided a pump that is configured to administer an infusion therapy using an invention medication delivery container by expelling medications in the flexible bag of the invention container from the bag and delivering the medications to an infusion site. The pump provides improved administration of infusion therapy which is particularly advantageous for reducing errors, infections and other complications associated with manual infusion techniques.

The pump can be configured to administer an infusion therapy using an invention medication delivery container. The pump can be further configured to specifically interface with an invention medication delivery container (hereinafter, “bag”) that is compartmentalized to contain multiple, separate medication solutions, and to deliver the solutions in a sequential, rate-controlled manner. Accordingly, invention pumps comprise a structure for applying constant force to a bag in a manner that sequentially activates chambers within the bag so that fluid contained therein is driven out through one or more conduits associated with each chamber, and into an intravenous (i.v.) drug delivery system (e.g., an administration set comprising microbore tubing that is attachable to a standard i.v. needle).

In accordance with yet another embodiment of the present invention, there is provided a housing for receiving and retaining an invention medication delivery container (bag), as described herein, during the pumping operation. The housing further contains the structure for applying constant force to the bag.

The housing (e.g., a pump housing as described herein) can be configured to specifically receive a particular type of bag. This configuration can comprise any structure(s) that will serve to hold a specific bag in operative relationship with the mechanism for constant force. As used herein, “operative relationship with the mechanism for applying force” means that the bag is retained in a manner that allows the mechanism for applying force to activate bag chambers in the intended sequence, without displacing the bag so as to prevent correct operation. For example, the housing can include positioning pins that match holes in a medication container bag, fasteners (e.g., hook and loop, snaps, buttons, zippers, or the like) that mate with counterparts on the bag, or the like. In a particular embodiment, the housing is further configured to receive a manifold attached to the bag. By employing sufficient structure to retain the manifold, the bag is further secured.

In a preferred embodiment, the mechanism for applying force to expel liquid from the container contemplated for use in the practice of the present invention is a pump with a constant force spring. However it should be understood that other structures for applying force may be substituted therefor, including a roller attached to a constant force spring, a motor-driven roller, or the like. Each such mechanism will require a different housing configuration to retain the structure and to maintain it in operative relationship with the bag during the pumping or activation process. All such housing configurations are contemplated as within the scope of the present invention.

Because it is often desirable to further control the rate at which force is applied by the constant force spring, in one embodiment, invention pumps comprise an energy absorption device. Any suitable energy absorption device may be employed. Energy absorption devices contemplated for use in the practice of the present invention include both mechanical and electrically operated devices. Mechanical devices include watch-type gear assemblies (as further described herein), watch escapements, an air resistance device, a resistance rack, an eddy current gear, a viscous damper, and the like. As used herein “watch-type gear assembly” means an assembly comprising a plurality of interconnected toothed cogs or gears that operate, in a manner known to those of skill in the art, to absorb energy by rotating and also to modulate the rate of rotation in a predictable manner. The energy absorption device can be secured to the constant force spring at its hub. Thus, the constant force spring has a maximum rate it can travel as determined by the strength of the spring, the configuration of the bag, and the amount and nature of the fluid contained in the bag. The energy absorbing device then further limits the rate at which the constant force spring can travel (i.e., work).

The invention pump can further comprise an activating mechanism for charging or cocking the mechanism for applying force to the container. This can be accomplished in a variety of ways depending on the exact type of activating mechanism employed. In an embodiment where a constant force spring is used, the charging mechanism will act to translate energy input by the user into stored energy in the constant force spring. This can be accomplished in a variety of ways, depending on the exact type of constant force spring employed. In one embodiment, wherein the constant force spring comprises a coiled leaf of metal or other suitable material attached to a hub at the center of the coil, the charging mechanism is attached to the hub. The other end of the spring is fixed to the pump housing proximal to one end of the housing. In this manner, force can be applied to the center of the hub and directed away from the fixed end of the spring, thereby causing the spring to unroll. It is presently preferred that the hub of the spring protrude from either side of the spring so that the hub can be captured in a track or like structure for retaining and guiding the travel of the constant force spring. In this manner, the travel of the spring can be controlled during charging and in performing its work. It is even more preferred that the hub have additional structure for facilitating even retraction of the spring (i.e., so that one side is not unrolled faster than the other). This can be accomplished in a variety of ways, including employing a toothed gear and track assembly, as further described herein, or the like. The hub, gear and track assembly serves an additional function of providing an attachment point for the energy absorption device described herein, as well as a means to control the forward (i.e., work producing) travel of the spring.

Charging mechanisms contemplated for use in the practice of the present invention can include a force transmission structure suitable for pushing or pulling the hub of the spring in the intended direction (i.e., away form the fixed end of the spring). Suitable force transmission structures include chains, belts, rods or the like, if the hub is to be pulled; and rods, or the like if the hub is to be pushed. More specifically, charging can be accomplished by employing a crank, a pneumatically operated mechanism, a plunger, a slide, or the like. It is presently preferred that the force transmission structure be connected to a mechanism for providing a mechanical advantage to the user, as the energy required to charge the constant force spring can be substantial. A mechanical advantage can be provided in the form of a lever mechanism, a multi-stage cocking mechanism, or the like. A multi-stage cocking mechanism allows partial cocking or charging of the constant force spring during each stage of the cocking. In this manner, the often substantial force required to charge the constant force spring can be parceled out over several operation stages, thereby making cocking easier than if a single stage mechanism where employed.

Advantageously, the pump will also comprise an indicator such as a wheel, or the like to indicate the progress of infusion of the medication to the patient. The indicator can interface with the activating mechanism and any associated gearing to provide a true indication of the progress made by the activating mechanism. In a preferred embodiment, the indicator is geared in a manner to amplify the progress of infusion.

In one embodiment, described with reference to FIGS. 16, 17, and 18, the medication delivery pump 110 of the invention includes a receptacle 112 for receiving a bag of the medication delivery container. A spring assembly 114 in the receptacle rolls up and compresses the bag at a maximum rate controlled by an energy absorbing device 116 in the form of a timer assembly. Medications in the chamber(s) of the bag are expelled from the bag through a suitable exit structure, e.g., a manifold assembly, and into an administration set attached to the manifold assembly. The administration set delivers the medications to an infusion site. The pump, in combination with the container, provides improved administration of infusion therapy which is particularly advantageous for reducing errors, infections and other complications associated with manual infusion techniques.

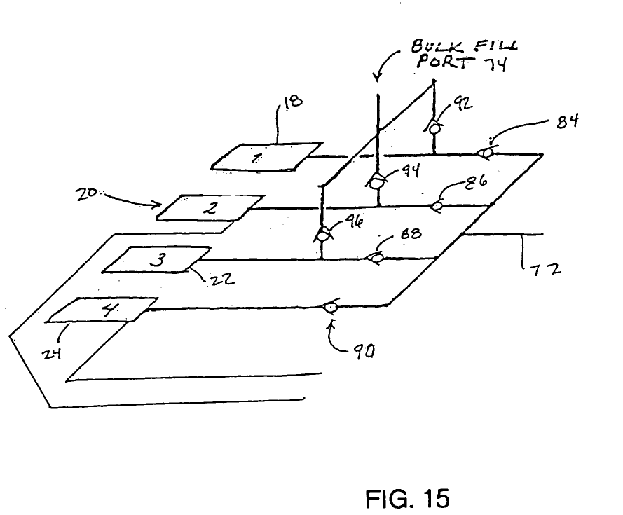

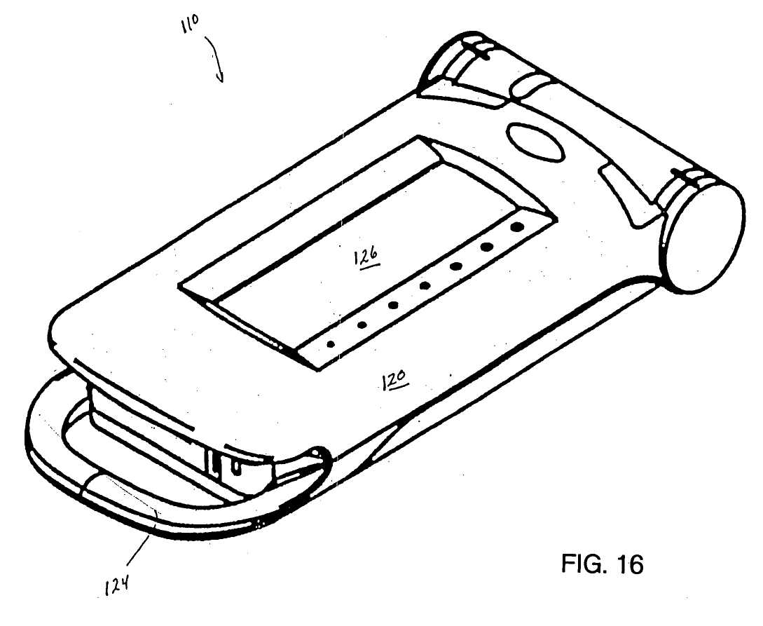

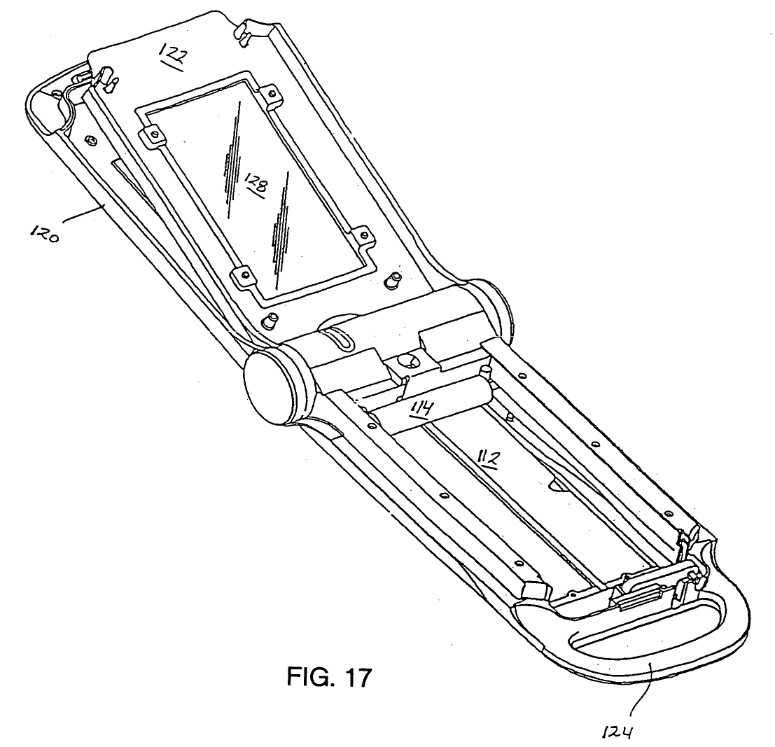

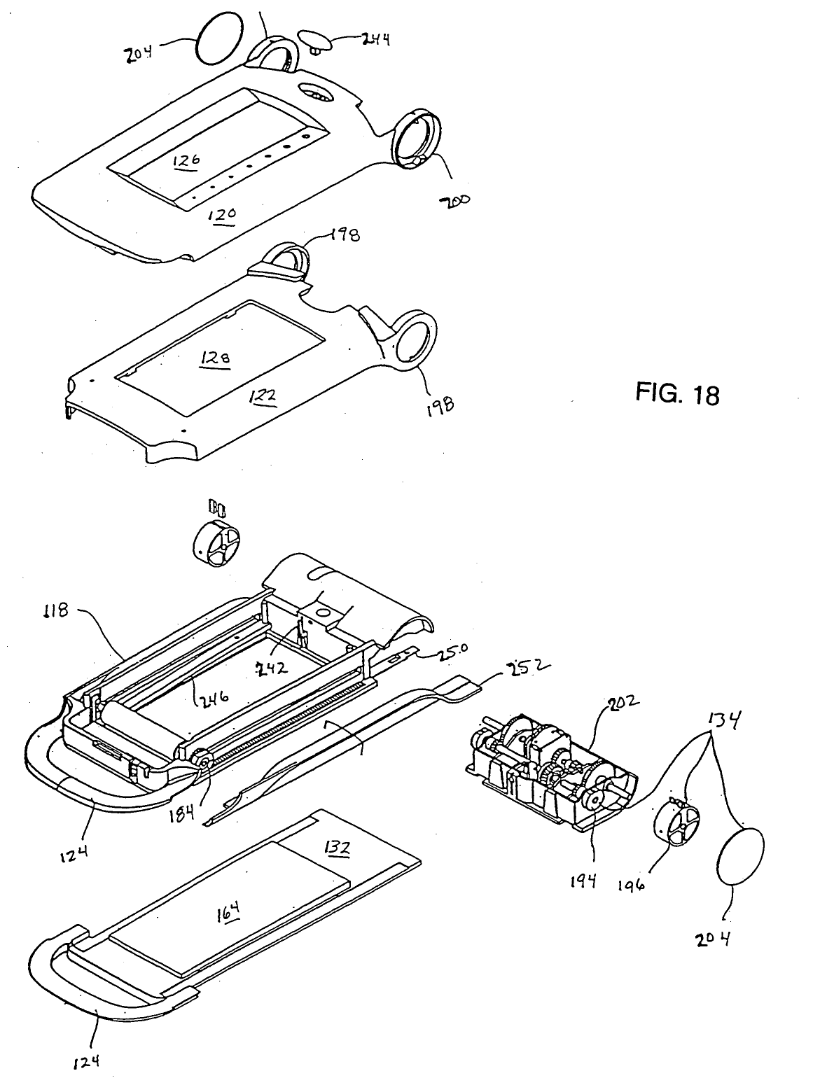

FIG. 18 illustrates a pump housing that includes a base 118 and a pair of cover doors, 120 and 122, respectively. The cover doors are opened to provide access to the container receptacle and to charge the spring assembly. In embodiments where a two-stage, door-operated charging mechanism is not employed, a single door can be used. The pump housing, illustrated in FIGS. 16, 17 and 18, preferably includes a handle 124 for carrying the pump and to assist in holding the pump as the first and second cover doors are opened to charge the spring. The cover doors also optimally include a window or opening, 126 and 128, in each cover to allow viewing of the spring assembly and the bag in the receptacle. The base includes a container receptacle, a mechanism for applying constant force, such as a spring assembly 114, optional access points such as a bottom cover 112, a charging assembly 134 and an energy absorption device 116.

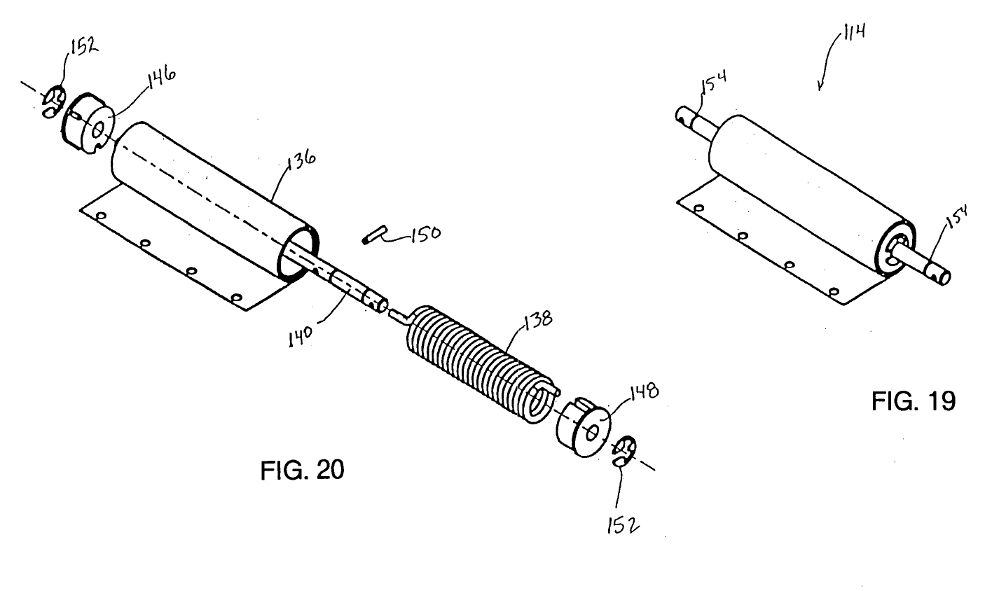

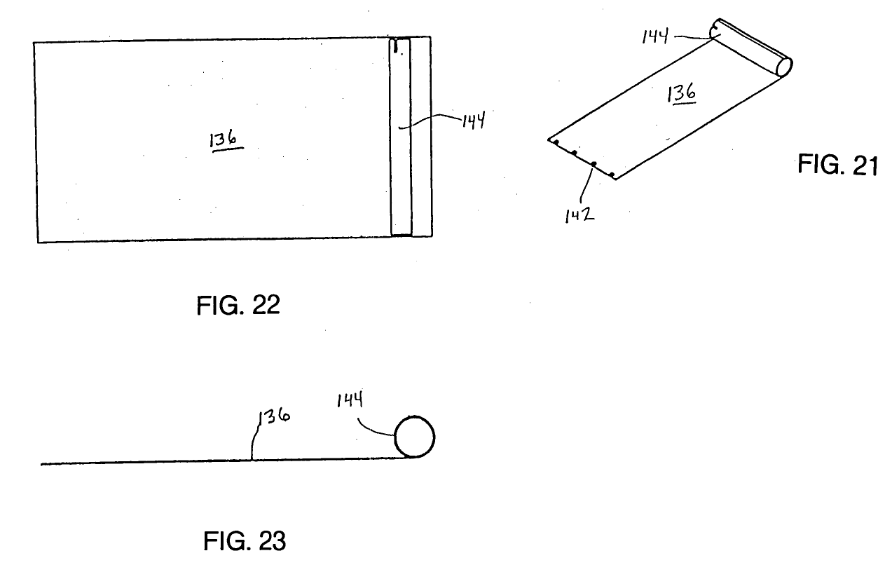

With reference to FIGS. 19-20, the spring assembly 114 includes a constant force pump spring mechanism 136, such as a torsion spring 138, for keeping the constant force spring wound to provide appropriate radial force, and a pump spring shaft 140. The constant force spring, shown in FIGS. 21-23, is formed of any suitable material having resilient properties, e.g., a sheet of steel. The pump spring preferably has a structure such as holes 142 at one end for convenient attachment to the base 118. Those of skill in the art recognize that other structures for attachment can be employed such as a clamp or adhesive. A drum 144 is suitably attached, e.g., welded, to the other end of the pump spring. At rest, the pump spring is completely coiled. The torsion spring has one end connected by suitable means, e.g., a first bushing 146 to the drum inside of the pump spring. The other end of the torsion spring is connected to the shaft by a suitable device, e.g., a second bushing 148. In order to prevent the second bushing from rotating on the shaft, the bushing is attached to the shaft by a pin 150, or other suitable structure. The first and second bushings are held in place on the shaft by respective retention devices such as nuts, or, as depicted in FIG. 20, first and second e-rings 152 that engage slots on the shaft. As discussed in more detail below the torsion spring is one device that can be employed to provide radial tension on the pump spring as it compresses and rolls up the bag.

As shown in FIG. 24, the base 118 includes a frame 156 and structure (e.g., slots 172 and pair of racks 158) for retaining the pump spring hub and guiding the travel of the pump spring. The frame has at least four sides that form the sides of the container receptacle 112. At a convenient location, e.g., at a front side of the frame, is a handle 124 and a side opening to a tube exit 160. Adjacent the tube exit is a recess configured to receive a manifold assembly if one is present on the container. In the depicted embodiment, the pump spring assembly 114 has one end (opposite the drum end) attached using a plate 163 to the frame adjacent to the front side. Any manner suitable for attaching the pump spring to the housing base can be employed in the practice of the present invention.

It can be advantageous to access the components of the pump for purposes such as maintenance or adjustment; accordingly, in one embodiment of the present invention, the housing can have one or more removable portions to provide the needed access. For example, a bottom cover 132 can be removably secured to the bottom of the frame. The housing is sized to accommodate the pump spring in any state of charging. In one embodiment, the bottom (or bottom cover, when employed) has an inclined plate 164 (FIG. 18) that is tapered to accommodate an increasing spring diameter as the spring rolls up the bag. Accommodations are also included for the energy absorption device and the charging assembly. In the depicted embodiment, at the rear side of the frame is a compartment 166 for attaching the charging assembly and the timing assembly. As with other key components of the pump, it is advantageous to provide access to these components for maintenance. A window 168 is preferably provided into the compartment for viewing an indicator device, such as a wheel 170, that indicates the rate of movement of the pump spring. On two long sides of the frame are structures to receive the hub of the spring (or roller); contemplated structures are exemplified by slots 172 and adjacent ledges 174. The racks 158 are mounted on the respective ledges, or are otherwise accommodated within the housing in alternative embodiments. Side covers 252 may be employed to cover the spring gear and rack.

The constant force pump spring assembly can be retained in the housing in a variety of ways. Referring to the embodiment shown in FIG. 24, the spring assembly 114 fits in the bottom of the container receptacle with the shaft extending through the slots 172 in the long sides of the frame 156. Located at each end of the spring shaft 140 are suitable drive structures, e.g., first and second gears 176, respectively. Other drive structures such as a bearing and race assembly, or the like, can be employed in the alternative. Structures for further retaining the spring include two horizontal slides or guide blocks 178 which are on the shaft between each gear and the pump spring and are configured to slide along the respective slots while allowing the shaft to rotate. Each gear is held on the shaft by suitable attachment devices, e.g., a pin 180 and an e-ring 182. Each gear engages the corresponding rack 158 to rotate the shaft as the spring assembly slides in the slots.

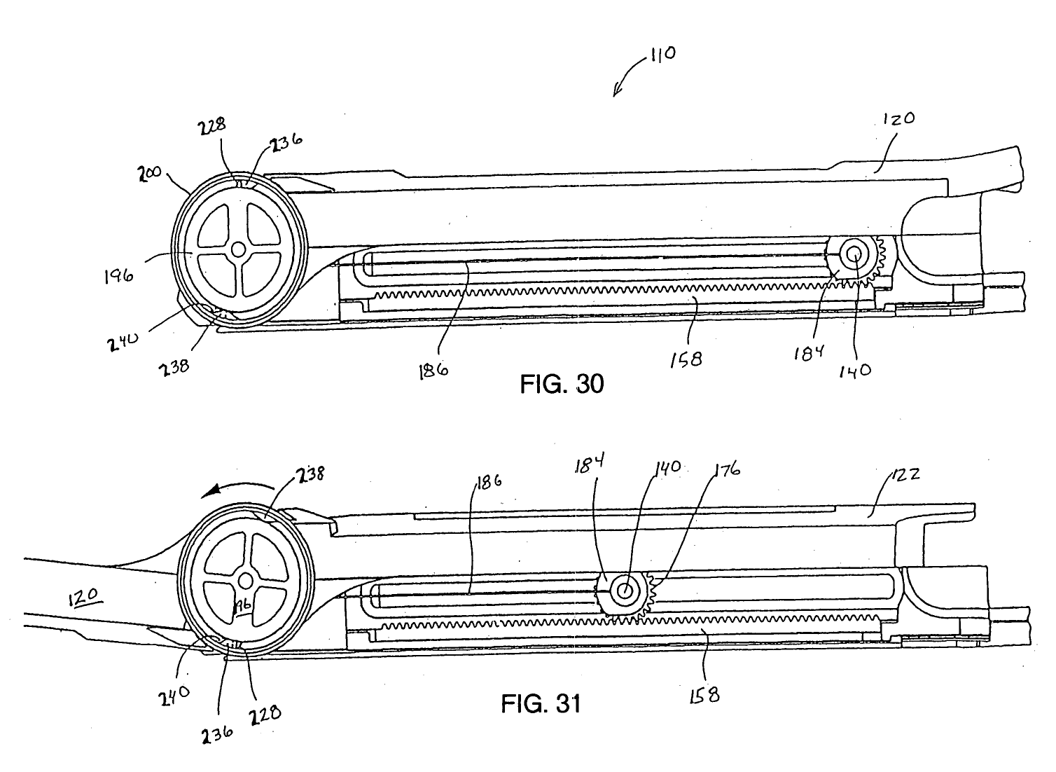

A mechanism for charging the constant force spring can be attached to the spring hub for pulling or pushing the hub away from the fixed end of the spring. In one embodiment, the charging mechanism is coupled to the spring hub by a belt assembly. In this embodiment, the hub will have sufficient structure, either as part of the hub, or attached to the hub, to facilitate secure attachment of the charging mechanism to the hub. For example, at each end of the shaft, adjacent to the respective gear (if employed), can be a belt hub 184 (FIG. 18). Each belt hub is attached to one end of a belt 186 (FIG. 30) formed of suitable material, e.g., a spring of steel. The other end of each belt is attached to the charging mechanism assembly 134. In this embodiment, the belt performs a dual purpose, i.e., both charging and rate control. The belt is also attached to the energy absorption device which controls the maximum rate at which the constant force spring can work. Thus, the energy absorption device serves to hold back, via the belt, forward progress of the constant force spring.

A constant force spring 136 has a tendency to roll up the bag 188 (FIG. 35) faster than the fluid may be expelled from the chambers because the hub of the spring is of fixed diameter, while the diameter of the spring changes as it rolls up. As a result, the tension on the spring can vary (i.e., lesser in the early portion of the pumping process and greater during the later portion of the spring travel), thereby allowing the spring to roll over fluid-containing chambers in the bag in the early portion of the spring travel, while possibly stalling due to increased tension in the later portion of the spring travel. Accordingly, a tension force may be applied to the end of the constant force spring that is distal to the hub in order to maintain the spring in a tightly coiled configuration in the early stages of the spring travel while lessening the tension in the later stages of the spring travel. It is presently preferred to have the distal end of the constant force spring fixed. Thus, in the presently preferred embodiment, a structure is provided to allow for relative motion between the hub and the constant force spring so that the constant force spring is tightened during the early stages of its travel and slackened during the later stages of its travel. The force provided by the energy absorption device can be translated to the constant force spring, while still allowing the relative motion between the hub and the spring by employing a tensioner mechanism as exemplified in FIG. 20. This figure depicts a torsion spring 138 that is internal to the drum 144. As force is applied to the hub, it is transferred to the tension spring which discourages or prevents the constant force spring from rolling over chambers of the bag that still contain fluid.

In the embodiment depicted in the FIGS. 16-18, the position of an uncharged constant force spring assembly 114 is at a front or handle end of the container receptacle 112. Mechanical energy is stored in the pump spring 136 using a charging assembly 134. As discussed in more detail below, the charging assembly uses a ratchet mechanism coupled to the two cover doors, 120 and 122. Although other charging mechanisms may be employed in the practice of the present invention, a two-door ratchet mechanism is presently preferred because it reduces the force required to be applied to open a cover door during charging of the pump spring. The pump spring is pulled back a substantial portion of the distance across the receptacle, e.g., 25-75%, by opening the outer cover to an open position. The pump spring is pulled back the remaining distance by opening the inner door. Of course, other charging mechanisms can be employed, such as a wind up mechanism comprising a reduction gear, an external handle attached to a reduction gear or ratchet mechanism, or the like.

The charging assembly 134 includes the belts 186, two belt drums 144 (FIG. 18), charging disks 194, and hub rings, 198 and 100, on the cover doors, respectively. It is presently preferred, for even application of force to the spring, that the charging assembly is substantially symmetric with similar components along both sides of the pump. The components on each side of the charging assembly are coupled by a gear box assembly 202. For cosmetic and protective purposes, the charging assembly can be covered on both sides by end caps 204.

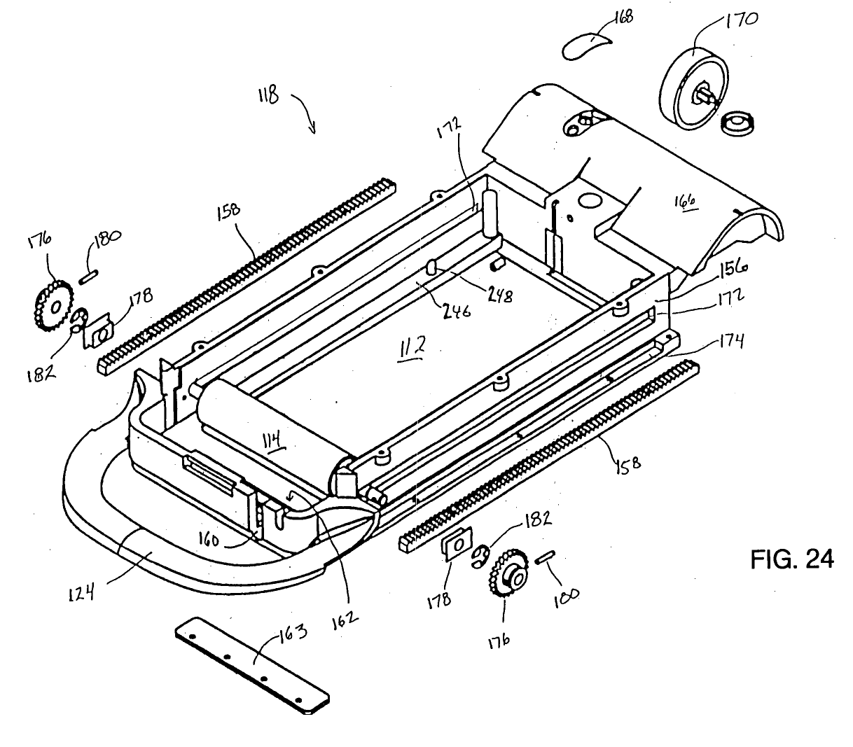

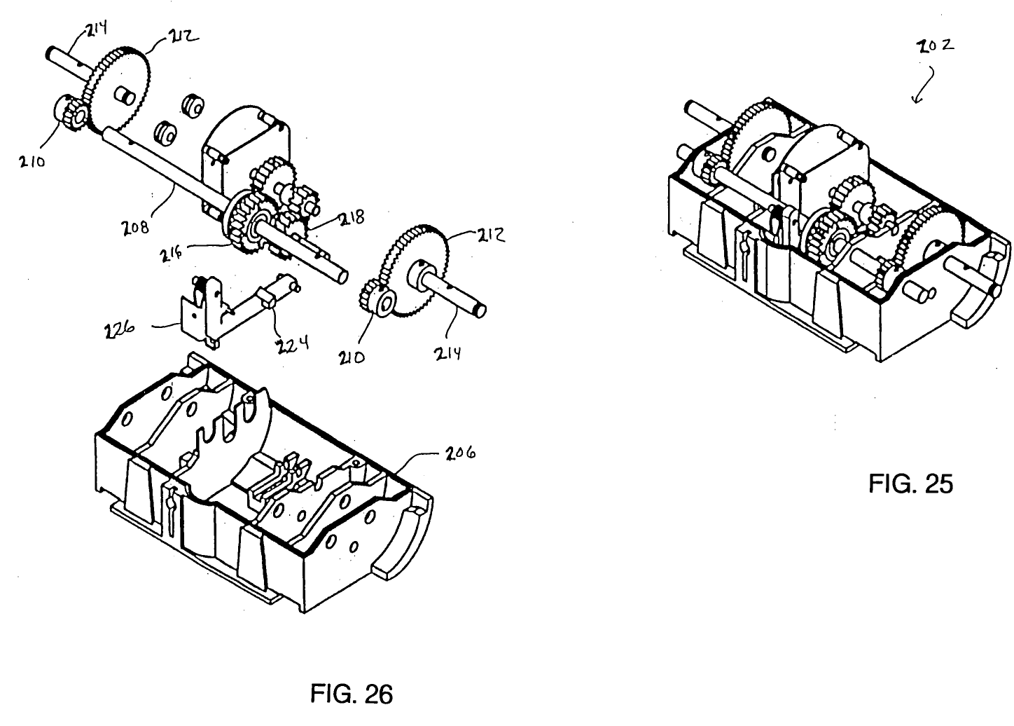

The gear box assembly 202, shown in FIGS. 25-26, includes a gear box 206, and associated gearing to transmit force from a charging interface such as a handle, or the like, to the constant force spring. In one embodiment, the associated gearing includes a link shaft 208, first and second spur gears 210, and first and second charging gears 212 on first and second charging shafts 214, respectively. The spur gears and the charging gears will have an appropriate gear ratio for ease of operation. The ratio will, of course vary with the size of the pump apparatus and the nature of the pump spring. Presently, a ratio of approximately 3:1 is preferred. The belt drums (FIG. 18) are attached to the respective ends of the link shaft. The energy absorption assembly also resides in the gear box.

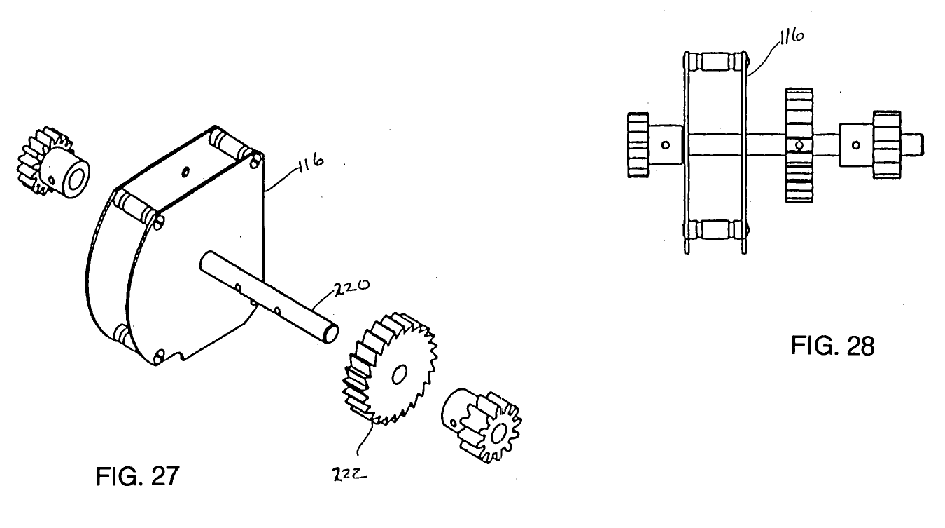

The energy absorption device/assembly 116, shown in FIGS. 27-28, controls the maximum rate at which the spring 136 may travel and compress the bag 188. Because the energy absorption assembly and the charging mechanism are both attached to the constant force spring, it is desirable to be able to disengage the energy absorption assembly during charging. Accordingly, in one embodiment, the link shaft 208 between the energy absorption assembly and the gear box assembly 202 includes a clutch assembly 216 that disengages the energy absorption assembly during charging of the pump spring. An idler gear couples the energy absorption assembly to the clutch assembly. On energy absorption assembly shaft 220 is a ratchet gear 222 that may be engaged by a start pawl 224 of the start/stop mechanism 226 (FIG. 26) to permit and halt rotation of the energy absorption assembly shaft and thus start and stop movement of the pump spring 136. Once a chamber of the bag is under compression, the fluid therein generates back pressure on the spring as it winds up on the shaft. The back pressure may limit the speed at which the spring travels. Thus, the energy absorption assembly's principle function is to limit the spring's maximum rate of travel, however, there likely will be times when the rate of spring travel is effectively limited by the fluid back pressure rather than the energy absorption device.

A charging disk 194, shown in FIG. 18, can be attached to the outside end of each charging shaft 214. When a two stage charging mechanism is employed, the charging disk has two catch mechanisms such as spring loaded pawls, 228 and 230, or the like. The first catch is engaged during the initial stage of the charging operation and the second catch engages during the second stage of the charging operation. When pawls are employed, at least the inner pawl has a tip beveled on one side so that a corresponding structure (e.g., the ramped tooth described below) on the hub ring (or its equivalent) can smoothly engage the pawl, while still providing a positive lock (when the non-beveled side of the pawl engages the ramped tooth). It is desirable that the shaft and slot are configured such that the inner pawl is depressed when the outer pawl is depressed; however, the outer pawl is not depressed when the inner pawl is depressed. Thus, in one embodiment, the outer pawl 228 includes a shaft 232 that engages a slot 234 on the inner pawl 230, thereby facilitating the desired operation.

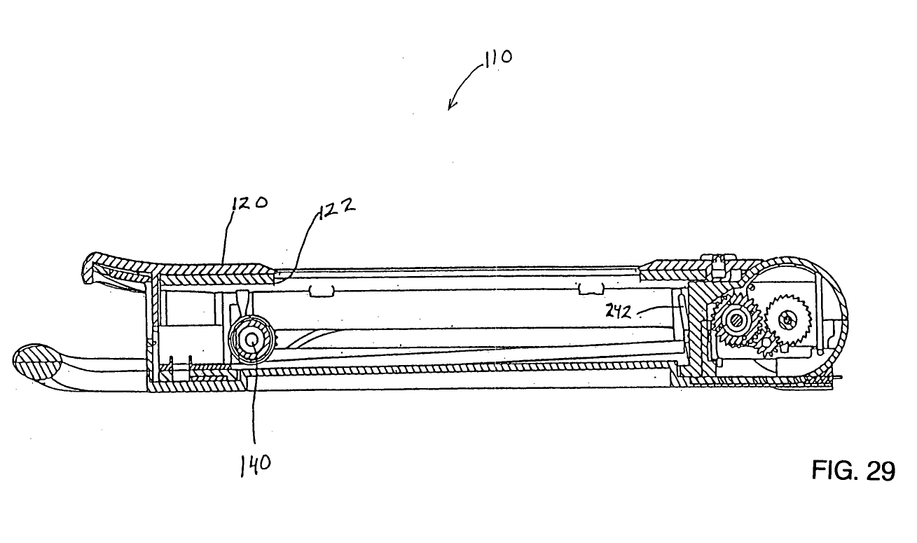

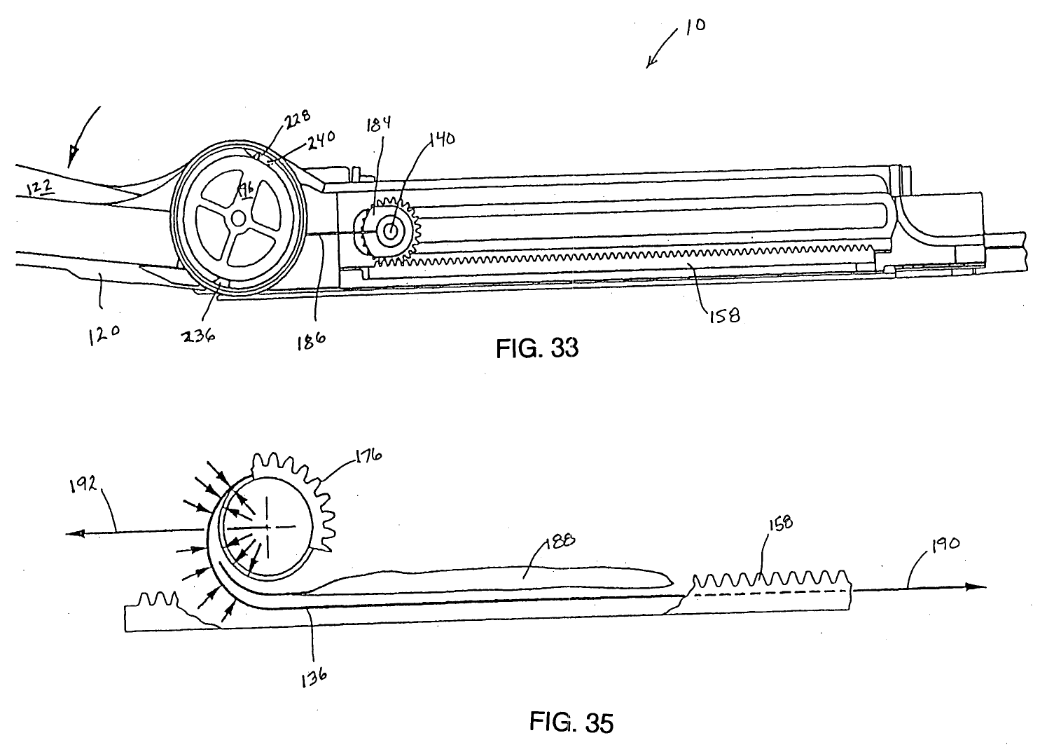

The pump spring charging operation will now be described with reference to FIGS. 29-33. The uncharged pump is shown in FIGS. 29-30. In this embodiment, the pump spring 136 is at the handle end of the receptacle. The hub ring 200 of the outer cover 120 has a ramped tooth 236 and a bypass ramp 238. The ramped tooth has one side that is perpendicular to the circumference of the outer hub ring for engaging the outer pawl 228 of the charging disk during the first stage of the charging operation (i.e., by opening the outer door). Thus by opening the outer door, the outer tooth engages the outer pawl and partially rotates the charging disk, thereby partially charging the spring as shown in FIG. 31. The charging disk rotation is transferred to the belt drum 84 which winds up the belt 186 thus pulling back the spring shaft 140.

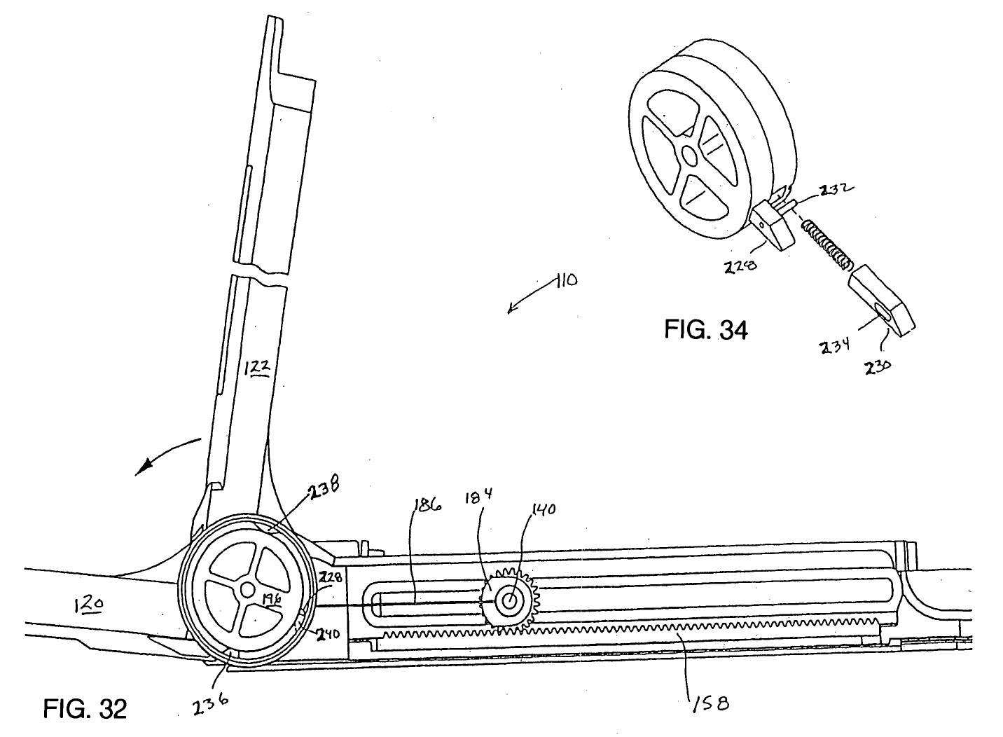

As shown in FIG. 32, the inner door further rotates the charging disk resulting in further pulling of the spring shaft as follows. The hub ring 198 of the inner cover 122 also has a ramped tooth 240 having a perpendicular side for engaging the inner pawl when the inner door is opened, thereby continuing the rotation of the charging disk to complete the charging operation. The inner tooth engages the inner pawl because, as the outer door is fully opened, the beveled side of the inner tooth rides over the beveled side of the inner pawl, depressing the inner pawl 230 (not shown) to clear the inner tooth. A start/stop pawl 224 (FIG. 18) in the receptacle is automatically engaged by a ratchet wheel 122 causing the gearbox assembly 202 to be locked into place. The bag 188 may now be placed in the pump 110 and both doors closed. A start button 244 (FIG. 18) can be activated after closing the doors. During discharge of the spring (i.e., during pumping operation), the bypass ramp 238 operates to depress the outer pawl (and, consequently, the inner pawl), thereby allowing the inner pawl to clear the inner tooth as the charging disk rotates back around in the opposite direction it rotated during charging.

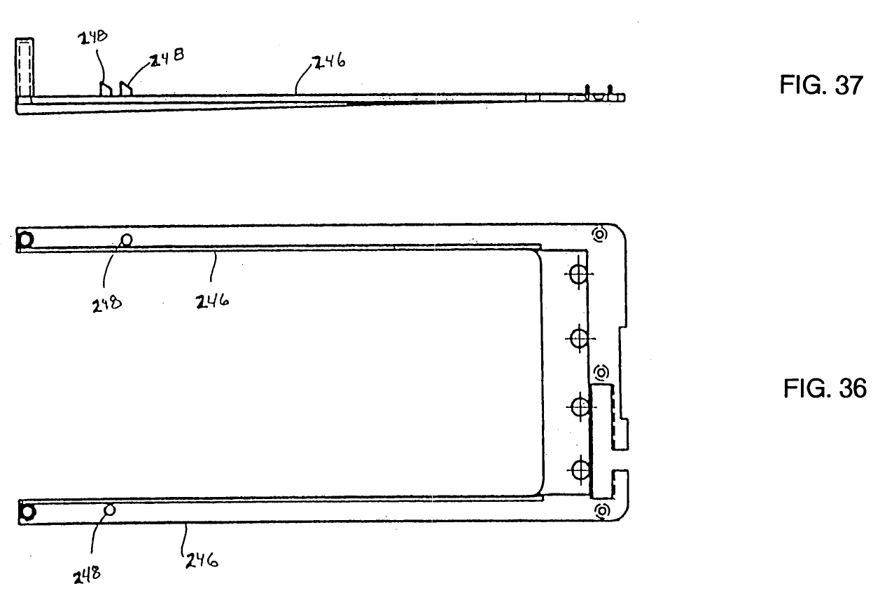

The pump may include a number of features for ensuring the correct administration of the desired infusion therapy. The receptacle may have two spring guards 246, shown in FIGS. 36-37, that prevent ready access to the edges of the constant force spring 136 which tend to curl up when the spring is in the charged position. Another optional, yet presently preferred feature is an internal structure, such as a set of pins 248 on the spring guard, that mate with the bag for correct positioning of the bag in the receptacle. The pins are designed so that the bag 188 will lift off the pins as it rolls up into the spring. The pins are offset from one another within the receptacle so that the bag can be easily placed in the receptacle in only one direction.

Interlocks can also be included so that the pump can only operate as intended. For example, a door interlock can be employed to prevent the inner door from being opened until the outer door is fully opened. The pump may also have a start button interlock 250 (FIG. 18) that detects if either of the covers are opened during the infusion. The start button engages the start/stop pawl when the door is closed, allowing the pump to operate. As a preferred safety feature, when the outer door is opened, the start button disengages from the start/stop pawl, and the pump is stopped. If the inner door is opened, the infusion is aborted. Further, the start button interlock also disables the start/stop button so that the spring motion cannot be reinitiated without recharging the pump. Aborting the infusion and disabling the start/stop button prevent improper administration caused by user interference with the bag configuration in the receptacle.

The fit and form of the pump with the doors closed is shown in the embodiment exemplified by cross-sectional diagram of FIG. 29. Corrosion resistant material may be used for those parts that may come in contact with fluids. The frame of the housing may be constructed of suitable corrosion resistant materials of sufficient rigidity, etc., e.g., polybutylene terephthalate (PBT) or similar polymer material. The rack and gears may be constructed of a metal such as brass, or the like, or a plastic material of suitable strength.

The medication delivery pump automates a number of labor steps typically used to administer multiple intravenous solutions in the proper volumes and in the proper sequence with minimal user interaction. Further, in a preferred embodiment, the pump is a mechanical device which does not require electrical energy nor software to correctly implement an infusion therapy.

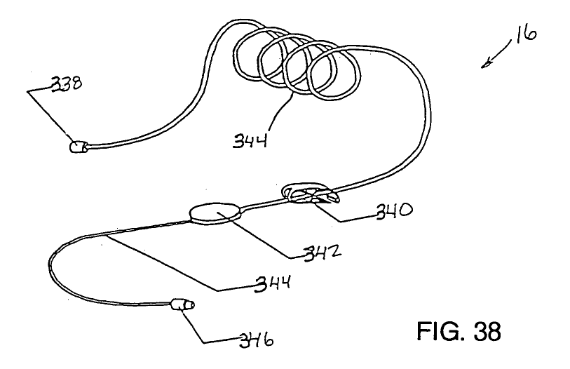

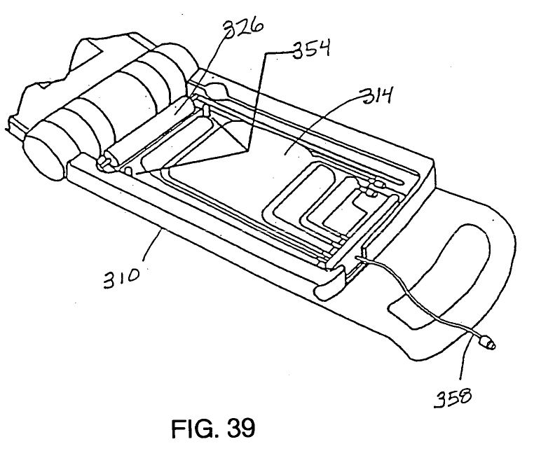

An administration set, as described hereinabove, is optionally provided in one embodiment of the present invention and can optionally be included in the invention medication delivery system. The embodiment of the administration set shown in FIG. 38 includes male and female luer connectors (338 and 346, respectively), or other equivalent attachment structures, a tubing clamp 340, an air-eliminating filter 342, a particulate filter (not shown), micro-bore tubing 346, and a flow restrictor (not shown). The tubing of the administration set may be composed of any biocompatible material such as a non-phthalate containing polyvinyl chloride (PVC) (i.e. non-DOP, dioctyl phthalate and non-DEBP, di-2-ethyl-hexyl-phthalate), or like tubing material which is commonly used in commercially available devices. The administration set may be connected to the bag by means of a standard male luer connector 348 on the bag that couples to the female luer connector of the administration set. The use of standard luer connectors provides assurance that the connection will be achieved easily and correctly. The air eliminating filter removes particulates larger than about 0.2 micron in diameter, and expels air in the fluid stream out of the air vent.

In another embodiment of the present invention there is provided a restrictor set for attachment to the distal end of the administration set. In this manner, the rate of fluid flow can be altered with the simple addition of a restrictor set, rather than by re-engineering the administration set. Of course, the maximum fluid flow rate will be determined by the configuration of the administration set, with fine-tuning to slower rates provided by the restrictor set.

The invention methods will now be described in greater detail by reference to specific, non-limiting embodiments as illustrated in FIGS. 38-40. Moreover, each of the embodiments of the various components described below need not necessarily be used in conjunction with the other specific embodiments shown.

In accordance with a specific embodiment of the invention methods, the user attaches the administration set (FIG. 38) to the bag, opens the two doors of the pump, thereby charging the activation mechanism, and places the bag inside a receptacle area within the pump housing (FIG. 39). The user then closes the doors of the pump, attaches the administration set to a patient's intravenous (i.v.) catheter site, and starts the activation mechanism, for example, by pushing a start button on the exterior of the housing. A mechanical spring (e.g., a constant force spring) within the pump sequentially compresses each of the bag's four chambers. The fluid within each chamber is sequentially expressed out of the bag, through the administration set, and into the patient. In a preferred embodiment, an indicator notifies the user when the infusion is complete. The indicator may be visual, audible, (e.g., a bell, or the like), tactile, or the like.

The medication delivery system is designed to be simple, safe, intuitive, and cost effective. Further, the system is designed to (1) reduce the need for supplies, (2) diminish manual manipulations and labor complexity, (3) decrease entries into the patient's TV catheter, and (4) ensure fluids will be administered in the proper volumes and in the proper sequence.

The invention medication delivery pump provides the advantage that it is a mechanical device which does not require electrical energy nor software to infuse the solutions in the correct volume, order, and flow rate. An activating mechanism such as a constant force stainless steel spring provides the mechanical energy to express the fluids as it compresses each solution chamber of the bag.

The solution pressures and infusion rates are determined by the system's configuration. A governing mechanism in the pump works to limit the maximum allowable speed of advance of the spring. When the rate of travel of the constant force spring exceeds the maximum rate allowed by the governor, the governor absorbs some of the spring energy to limit the speed of the spring's travel. The governor allows the spring to move over the entire distance of the pump at a minimum, predetermined amount of time. Thus, the pump generates predictable fluid pressures based on the volume of solution in each chamber. Using the predictable fluid pressures, the flow rate from the bag may be selectable using administration sets having predetermined tubing lengths and inner diameters. The continuous force by the spring on the bag, in combination with check valves in a manifold of the container, prevents the reverse flow of fluids from the administration set to the container.

In the embodiment where the pump comprises a two stage charging mechanism comprising inner and outer doors, the pump's outer door and inner door must be opened in order to place a filled bag inside the pump. The opening motion of the outer door and inner door is the mechanism by which the mechanical pump spring is pulled back to the start position. After the inner and outer doors are closed, the pump is ready to be started upon pushing of the “start” button. The cut out windows in the inner and outer door allow the user to observe the position of the spring as it moves in relation to the bag. Accordingly, the user is able to visually monitor the progress of the infusion.

The pump may be designed to separate the bag compartment or receptacle from most of the pump's moving parts. Corrosion resistant materials may be used for any parts that may come in contact with liquids. This attention to the physical design facilitates cleaning of the pump.

The flow of solution from each chamber is initiated due to a pressure build up caused by the pump spring compressing the filled chamber. As the pressure increases, a check valve in the manifold opens, allowing the fluid to flow from the chamber, down a fluid conduit, past the valve, and out through a single outlet tubing into the patient. When the solution is expelled from the chamber, a drop of pressure occurs which allows the valve to close. It is the opening and closing of the valves that governs the starting and stopping of solution flow from each respective chamber. The controlled rate at which the spring compresses the bag maintains the solution pressure below the typical maximum safe pressure for i.v. devices (i.e., catheters, luers, needles, and the like).

Features for filling and using the invention medication delivery system are described with reference to FIGS. 38 and 39. Bag 314 is shown with a fill port 360 for bulk filling of chambers 1, 2, and 3 (referenced by numbers 328, 330 and 334, respectively). The system also has two separate injection sites, 362 and 364, for chambers 330 and 334 to allow one to add additional solutions. The administration set 316 may be primed by filling the manifold 336 and the tubing 344 through the bulk fill port 360 while the clamp 340 is locked until the air in the tubing is eliminated through the air-elimination filter 342. Further, a hydraulic lock feature may be formed between the air filter and the check valves by filling the manifold assembly and the tube to a positive pressure great enough to prevent the valves from opening and allowing leakage from the chambers during storage, handling, or transport of the bag 314. The hydraulic lock may be overcome upon the application of a threshold pressure to the respective chambers or release of the pressure by opening the clamp.

The bag includes a shipping clamp 368 for preventing leakage of any solutions subsequent to filling. When the bag is inserted in the filling fixture, the clamp is released to allow filling. Conversely, when filling is completed, the shipping clamp is closed to prevent leakage of solution from the filled bag prior to use.

A filling fixture is a pharmacy tool used only in filling the chambers of the bag. By restraining chambers 1 and 3 with the interior walls of the filling fixture, the operator assures that the filling fixture provides a physical constraint to the bag 314 to assure that each of chambers 1, 2 and 3 is filled to the correct nominal fill volume. Thus, in use, the operator places the bag 314 into the filling fixture prior to initiating the fill. Once the bag is in the filling fixture, the shipping clamp on the bag, if provided, is opened and the operator bulk fills chambers 1, 2 and 3 of the bag through the bulk fill port 360 in one step, using standard pharmacy filling equipment and procedures.

For example, the operator may fill the bag with 10 mls in chambers 1 and 3, and 100 mls in chamber 2, by setting the standard pharmacy filling equipment to dispense 120 ml. The fluid will flow into the bag, filling chambers 1 and 3 to 10 mls. The filling fixture will constrain chambers 1 and 3 to this volume and the remainder of the fluid (100 mls) will flow into chamber 2. When the bag chambers 1, 2 and 3 are filled, each to the desired volume, the operator removes the bag from the filling fixture. The bag is now ready to have solution added to chambers 2 and 4 via the injection sites, 362 and 364, respectively, as required by the operator. The chamber 2 and chamber 4 injection sites are accessed via standard pharmacy filling equipment and procedures. Upon completion of filling, the bag is ready for insertion into the pump 310 for delivery of the solutions.

The invention will now be described in greater detail by reference to the following non-limiting example.

### EXAMPLE

The following example illustrates flow from the invention medication delivery system using a four-chambered bag having the following chamber fill volumes:

| | | |
|---|---|---|
| Chamber 1 | 5-10 | mls. |
| Chamber 2 | 100-120 | mls. |
| Chamber 3 | 5-10 | mls. |
| Chamber 4 | 5 | mls. |

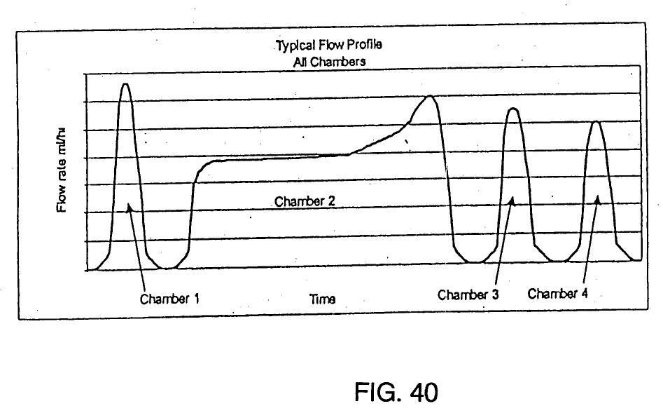

A typical flow profile of fluid flow from the four bag chambers over time is shown in FIG. 40. The larger chamber 2 has a relatively flat administration profile until the end of the administration at which time the flow peaks and then rapidly drops to zero. The smaller chambers similarly exhibit peaked administration profiles. The flow rate may be selected by selecting the inner diameter and the length of the micro-bore tubing in the administration set. A smaller inner diameter or a longer length of tubing reduces the flow rate and increases the administration time. Conversely, a larger inner diameter or a short length of tubing increases the flow rate and decreases the administration time.

While the invention has been described in detail with reference to certain preferred embodiments thereof, it will be understood that modifications and variations are within the spirit and scope of that which is described and claimed.

What is claimed is:

1. A method of providing a therapy regimen to a patient, said therapy regimen comprising the delivery in a predetermined sequence of a plurality of fluids from an integral container to said patient, said integral container comprising said plurality of fluids contained within separate non-fluidly connected chambers, the method comprising:
delivering said plurality of fluids from said separate non-fluidly connected chambers to said patient in said predetermined sequence.

2. The method of claim 1, wherein said plurality of fluids are delivered in a predetermined sequence by exerting positive pressure on said separate non-fluidly connected chambers, whereby said fluids are expressed from said chambers in said predetermined sequence.

3. The method of claim 2, wherein said positive pressure is created by compression of said separate non-fluidly connected chambers in a predetermined sequence.

4. The method of claim 1, wherein said plurality of fluids are delivered in a predetermined sequence by exerting negative pressure on said separate non-fluidly connected chambers, whereby said fluids are extracted from said chambers in said predetermined sequence.

5. The method of claim 4, wherein said negative pressure is created by pumping said fluids from said separate non-fluidly connected chambers in a predetermined sequence.

6. The method of claim 5, wherein negative pressure is exerted on each said separate non-fluidly connected chamber by a separate pump.

7. The method of claim 6, wherein each said separate pump is controlled by a programmable interface.

8. The method of claim 1, wherein said plurality of fluids are delivered in a predetermined sequence by gravity feed from said separate non-fluidly connected chambers, whereby said fluids flow from said chambers in said predetermined sequence.

9. The method of claim 8, wherein said plurality of fluids are delivered in a predetermined sequence by a differential hydrostatic head height in two or more separate non-fluidly connected chambers.

10. The method of claim 1, wherein said plurality of fluids flow from said separate non-fluidly connected chambers to a manifold comprising a separate input port in fluid communication with each said separate non-fluidly connected chamber and at least one common port, whereby said fluids flow to said common port in said predetermined sequence.

11. The method of claim 1, wherein flow from one or more of said separate non-fluidly connected chambers is controlled by valves.

12. The method of claim 1, wherein flow from one or more of said separate non-fluidly connected chambers is controlled by an active control device.

13. The method of claim 1, wherein flow from one or more of said separate non-fluidly connected chambers is controlled by a passive control device.

14. The method of claim 1, wherein two or more chambers in said integral container become fluidly connected prior to or during delivery of said plurality of fluids, whereby a single chamber is formed.

15. A fluid delivery device, comprising:
an integral container comprising a plurality of fluids contained within separate non-fluidly connected chambers;
wherein said fluid delivery device is configured and arranged to deliver said plurality of fluids from said separate non-fluidly connected chambers to said at least one common port in a predetermined sequence.

16. The fluid delivery device of claim 15, further comprising a manifold comprising a separate input port in fluid communication with each said separate non-fluidly connected chamber and at least one common port.

17. The fluid delivery device of claim 15, further comprising one or more pumping elements.

18. A medication delivery system, comprising:
a bag having:
at least one chamber containing a medication fluid, and a manifold; and
a pump having an activating mechanism configured to activate said chamber(s) to dispense said fluid from the bag.

19. A fluid delivery container, comprising:
a bag having at least one fluid chamber; and
structure for minimizing pressure drop between the chamber and an associated conduit upon the application of pressure to the chamber.

20. A fluid delivery pump, comprising:
a structure for sequentially applying constant force to compress a flexible fluid container from a first end towards a second end of said container; and
an energy absorption device coupled to the structure for sequentially applying constant force for limiting the maximum rate at which said structure compresses the fluid container.
